(19) United States
(12) Patent Application Publication
(10) Pub. No.: US 2011/0143994 A1
(43) Pub. Date: Jun. 16, 2011

(54) COMPOSITIONS AND METHODS FOR TREATMENT OF AUTOIMMUNE AND ALLERGIC DISEASES

(76) Inventor: Nils Lycke, Vastra Frolunda (SE)

(21) Appl. No.: 12/674,497

(22) PCT Filed: Dec. 15, 2008
(86) PCT No.: PCT/SE2008/051454

§ 371 Date: Feb. 22, 2010

(30) Foreign Application Priority Data
Dec. 19, 2007 (SE) 0702825-1

(51) Int. Cl. A61K 38/16; C07K 19/00; A61P 19/02; A61P 3/10; A61P 11/06

(52) U.S. Cl. 514/1.7; 530/350; 514/7.3; 514/16.6; 514/1.1

(57) ABSTRACT

The present invention provides improved methods and compositions for treating and preventing autoimmune and allergic diseases. More specifically the invention relates to new immuno-modulating complexes which are fusion proteins comprising mutant subunits of bacterial endotoxins, a peptide capable of binding to a specific cellular receptor, and one or more epitopes associated with an autoimmune or allergic disease.

### FIELD OF THE INVENTION

The present invention relates to the fields of immunology and medicine. The present invention provides improved methods and compositions for treating and preventing autoimmune and allergic diseases. More specifically the invention relates to new immunomodulating complexes which are fusion proteins comprising mutant subunits of bacterial endotoxins, a peptide capable of binding to a specific cellular receptor, and one or more autoantigenic or allergy-provoking epitopes associated with an autoimmune or allergic disease.

### BACKGROUND

### Autoimmune Disease and Modulation of the Immune Response

Autoimmune disease is any disease caused by immune cells that become misdirected at healthy cells and/or tissues of the body. Autoimmune disease affects 3% of the U.S. population and likely a similar percentage of the industrialized world population (Jacobson et al. Clin Immunol Immunopathol 84: 223-43, 1997). Autoimmune diseases are characterized by T and B lymphocytes that aberrantly target self-proteins, -polypeptides, -peptides, and/or other self-molecules causing injury and or malfunction of an organ, tissue, or cell-type within the body (for example, pancreas, brain, thyroid or gastrointestinal tract) to cause the clinical manifestations of the disease (Marrack et al. Nat Med 7: 899-905, 2001). Autoimmune diseases include diseases that affect specific tissues as well as diseases that can affect multiple tissues. This may, in part, for some diseases depend on whether the autoimmune responses are directed to an antigen confined to a particular tissue or to an antigen that is widely distributed in the body. The characteristic feature of tissue-specific autoimmunity is the selective targeting of a single tissue or individual cell type. Nevertheless, certain autoimmune diseases that target ubiquitous self-proteins can also affect specific tissues. For example, in polymyositis the autoimmune response targets the ubiquitous protein histidyl-tRNA synthetase, yet the clinical manifestations primarily involved are autoimmune destruction of muscle.

The immune system employs a highly complex mechanism designed to generate responses to protect mammals against a variety of foreign pathogens while at the same time preventing responses against self-antigens. In addition to deciding whether to respond (antigen specificity), the immune system must also choose appropriate effector functions to deal with each pathogen (effector specificity). A cell critical in mediating and regulating these effector functions is the CD4⁺ T cell. Furthermore, it is the elaboration of specific cytokines from CD4⁺ T cells that appears to be the major mechanism by which T cells mediate their functions. Thus, characterizing the types of cytokines made by CD4⁺ T cells as well as how their secretion is controlled is extremely important in understanding how the immune response is regulated.

The characterization of cytokine production from long-term mouse CD4⁺ T cell clones was first published more than 20 years ago (Mosmann et al. J Immunol 136: 2348-2357, 1986). In these studies, it was shown that CD4+T cells produced two distinct patterns of cytokine production, which were designated T helper 1 (Th1) and T helper 2 (Th2). Th1 cells were found to produce interleukin-2 (IL-2), interferon-γ (IFN-γ) and lymphotoxin (LT), while Th2 clones predominantly produced IL-4, IL-5, IL-6, and IL-13 (Cherwinski et al. J Exp Med 169:1229-1244, 1987). Somewhat later, additional cytokines, IL-9 and IL-10, were isolated from Th2 clones (Van Snick et al. J Exp Med 169:363-368, 1989) (Fiorentino et al. J Exp Med 170:2081-2095, 1989). Finally, additional cytokines, such as IL-3, granulocyte macrophage colony-stimulating factor (GM-CSF), and tumor necrosis factor-α (TNF-α) were found to be secreted by both Th1 and Th2 cells. Recently, it was reported that CD4+ T cells isolated from the inflamed joints of patients with Lyme disease contain a subset of IL-17-producing CD4+ T cells that are distinct from Th1 and Th2 (Infante-Duarte et al. J. Immunol. 165:6107-6115, 2000). These IL-17-producing CD4+ T cells are designated Th17. IL-17, a proinflammatory cytokine predominantly produced by activated T cells, enhances T cell priming and stimulates fibroblasts, endothelial cells, macrophages, and epithelial cells to produce multiple proinflammatory mediators, including IL-1, IL-6, TNF-α, NOS-2, metalloproteases, and chemokines, resulting in the induction of inflammation. IL-17 expression is increased in patients with a variety of allergic and autoimmune diseases, such as RA, MS, inflammatory bowel disease (IBD), and asthma, suggesting the contribution of IL-17 to the induction and/or development of such diseases.

There is ample evidence showing that suppressor T cells, now called regulatory T cells (Treg cells), suppress autoreactive T cells as an active mechanism for peripheral immune tolerance. It is, thus far, firmly established that Treg cells can be divided into two different subtypes, namely natural (or constitutive) and inducible (or adaptive) populations according to their origins (Mills, Nat Rev Immunol 4:841-855, 2004). In addition, a variety of Treg cell subsets have been identified according to their surface markers or cytokine products, such as CD4+ Treg cells (including natural CD4+CD25+ Treg cells, IL-10-producting Tr1 cells, and TGF-β-producing Th3 cells), CD8+ Treg cells, Veto CD8+ cells, γδ T cells, NKT (NK1.1+CD4−CD8−) cells, NK1.1−CD4−CD8− cells, etc. Accumulating evidence has shown that naturally occurring CD4+CD25+ Treg cells play an active role in down-regulating pathogenic autoimmune responses and in maintaining immune homeostasis (Akbari et al. Curr Opin Immunol 15:627-633, 2003).

Autoimmune disease encompasses a wide spectrum of diseases that can affect many different organs and tissues within the body. (See e.g., Paul, W. E. (1999) Fundamental Immunology, Fourth Edition, Lippincott-Raven, New York.)

Current therapies for human autoimmune disease, include glucocorticoids, cytotoxic agents, and recently developed biological therapeutics. In general, the management of human systemic autoimmune disease is empirical and unsatisfactory. For the most part, broadly immunosuppressive drugs, such as corticosteroids, are used in a wide variety of severe autoimmune and inflammatory disorders. In addition to corticosteroids, other immunosuppressive agents are used in management of the systemic autoimmune diseases. Cyclophosphamide is an alkylating agent that causes profound depletion of both T- and B-lymphocytes and impairment of cell-mediated immunity. Cyclosporine, tacrolimus, and mycophenolate mofetil are natural products with specific properties of T-lymphocyte suppression, and they have been used to treat systemic lupus erythematosus (SLE), rheumatoid arthritis (RA) and, to a limited extent, in vasculitis and myositis. These drugs are associated with significant renal toxicity. Methotrexate is also used as a “second line” agent in RA, with the goal of reducing disease progression. It is also used in polymyositis and other connective-tissue diseases. Other approaches that have been tried include monoclonal antibodies intended to block the action of cytokines or to deplete lymphocytes. (Fox, Am J Med 99:82-88, 1995). Treatments for multiple sclerosis (MS) include interferon β and copolymer 1, which reduce relapse rate by 20-30% and only have a modest impact on disease progression. MS is also treated with immunosuppressive agents including methylprednisolone, other steroids, methotrexate, cladribine and cyclophosphamide. These immunosuppressive agents have minimal efficacy in treating MS. The introduction of the antibody Tysabri (natalizumab), an alpha 4-integrin antagonist, as treatment for MS has been overshadowed by incidences of progressive multifocal leucoencaphalopathy (PML) in patients receiving the therapy. Current therapy for RA utilizes agents that non-specifically suppress or modulate immune function such as methotrexate, sulfasalazine, hydroxychloroquine, leuflonamide, prednisone, as well as the recently developed TNFα antagonists etanercept and infliximab (Moreland et al. J Rheumatol 28: 1431-52, 2001). Etanercept and infliximab globally block TNFα, making patients more susceptible to death from sepsis, aggravation of chronic mycobacterial infections, and development of demyelinating events.

In the case of organ-specific autoimmunity, a number of different therapeutic approaches have been tried. Soluble protein antigens have been administered systemically to inhibit the subsequent immune response to that antigen. Such therapies include delivery of myelin basic protein, its dominant peptide, or a mixture of myelin proteins to animals with experimental autoimmune encephalomyelitis and humans with multiple sclerosis (Brocke et al. Nature 379: 343-6, 1996; Critchfield et al. Science 263: 1139-43, 1994; Weiner et al. Annu Rev Immunol 12: 809-37, 1994), administration of type II collagen or a mixture of collagen proteins to animals with collagen-induced arthritis and humans with rheumatoid arthritis (Gumanovskaya et al. Immunology 91: 466-73, 1999; McKown et al. Arthritis Rheum 42: 1204-8, 1999; Trentham et al. Science 261: 1727-30, 1993), delivery of insulin to animals and humans with autoimmune diabetes (Pozzilli and Gisella Cavallo, Diabetes Metab Res Rev 16: 306-7, 2000), and delivery of S-antigen to animals and humans with autoimmune uveitis (Nussenblatt et al. Am J Ophthalmol 123: 583-92, 1997). Another approach is the attempt to design rational therapeutic strategies for the systemic administration of a peptide antigen based on the specific interaction between the T-cell receptors and peptides bound to MHC molecules. One study using the peptide approach in an animal model of diabetes, resulted in the development of antibody production to the peptide (Hurtenbach et al. J Exp Med 177:1499, 1993). Another approach is the administration of T cell receptor (TCR) peptide immunization. (See, e.g., Vandenbark et al. Nature 341:541, 1989). Still another approach is the induction of oral tolerance by ingestion of peptide or protein antigens. (See, e.g., Weiner, Immunol Today 18:335, 1997).

Mucosal tolerance refers to the phenomenon of systemic tolerance to challenge with an antigen that has previously been administered via a mucosal route, usually oral, nasal or naso-respiratory, but also vaginal and rectal. (Weiner et al. Annu Rev Immunol 12:809-837, 1994). Mucosal tolerance was discovered early in the 20th century in models of delayed-type and contact hypersensitivity reactions in guinea pigs, but the mechanisms of tolerance remained ill-defined until the era of modern immunology. The use of cell separation techniques, tests for production of cytokines and transgenic models in which antigen-specific T cells can be tracked in vivo have gradually elucidated mechanisms of mucosal tolerance (Garside and Mowat. Crit. Rev Immunol 17:119-137, 1997). It has become evident that antigen administration via mucosal routes can result in distinct types of tolerance depending on the route of administration and dose of antigen. For example, a high dose of oral antigen induces T-cell activation followed by deletion or anergy of responding T cells (Chen et al. Nature 376:177-180, 1995) analogous to parenteral administration of high-dose soluble antigen. This results in extinction of T cells specific to that antigen and unresponsiveness to subsequent antigen challenge, i.e. passive tolerance. In contrast, a low dose of oral antigen does not induce deletion or anergy but, when given repeatedly, induces a distinct type of immune response characterized by the appearance of regulatory-protective T cells, Treg cells, that secrete anti-inflammatory cytokines, i.e. active tolerance (von Herrath, Res Immunol. 148:541-554, 1997). These Treg cells usually belong to the class of CD4 (helper) T cells. Instillation of intact protein antigen onto the nasopharyngeal mucosa also induces Treg cells that are protective. In this case, both CD4 and CD8 T cells may be induced. Regulatory Treg cells induced after oral or intranasal antigen administration produce anti-inflammatory cytokines such as IL-4, IL-10 and TGF-β. To induce mucosal tolerance, antigen can also be given in the form of aerosol. Administration via these three routes, oral, intranasal and aerosol-inhalation, results in antigen uptake and presentation in different lymphoid compartments in each case. Accordingly, oral antigen is presented to T cells mostly in mesenteric lymph nodes and to some extent in Peyer's patches, intranasal antigen in deep cervical lymph nodes and inhaled antigen in mediastinal lymph nodes. Repeated exposure to antigen in each case is able to induce regulatory T cells, but the nature of these cells differs, depending on the route and form of antigen. While regulatory cells induced by oral antigen are CD4 T cells and express T cell receptors (TCR) consisting of αβ heterodimers, in the case of naso-respiratory antigen the regulatory cells can also be CD8 T cells expressing a γδ heterodimer TCR (i.e. γδ T cells). Some of these cells may also have a CD8 receptor that is an αα homodimer instead of the conventional αβ-heterodimer TCR. A majority of cells that carry the CD8αα and γδ TCR reside in skin or mucosal tissues.

Over the past decades, there has been a significant increase in both the incidence and prevalence of allergic disease in the western countries. Allergic rhinitis, is the most common of these diseases affecting 15-20% of the population. The allergic reaction is triggered by allergen-mediated cross-linking of specific IgE on the surface of mast cells and basophils leading to release of histamine and other mediators, causing an acute allergic reaction, followed by a late-phase reaction characterized by an influx of eosinophils, neutrophils and Th2 cells producing IL-4, IL-5 and IL-13.

Specific immunotherapy (SIT) is recognized as an effective treatment of allergic rhinitis Traditionally, SIT has been conducted by repeated subcutaneous administration of small amounts of specific allergen. Although this form of treatment can be an effective therapeutic option, concerns exist with the safety of this form of immunotherapy as well as with the difficulty of standardizing of the allergen extract used as vaccine. Consequently, there is strong interest in the development of alternative and novel treatments against allergic diseases. One of the approaches is the use of mucosal vaccines (Widermann, Curr Drug Targets Inflamm Allergy 4, 577-583, 2005). Other alternatives are based on the use of allergen derivatives with reduced or no allergenicity as vaccines (Vrtala et al. Methods 32, 313-320, 2004). These include allergens obtained by protein engineering and synthetic peptides representing immunodominant T-cells epitopes of allergens. For example, Ole e1 has been identified as the most relevant allergen of olive pollen (Wheeler et al. Mol Immunol 27,631-636, 1990).

Immune responses are currently altered by delivering polypeptides, alone or in combination with adjuvants (immunomodulating agents). For example, the hepatitis B virus vaccine contains recombinant hepatitis B virus surface antigen, a non-self antigen, formulated in aluminum hydroxide, which serves as an adjuvant. This vaccine induces an immune response against hepatitis B virus surface antigen to protect against infection. An alternative approach involves delivery of an attenuated, replication deficient, and/or non-pathogenic form of a virus or bacterium, each non-self antigens, to elicit a host protective immune response against the pathogen. For example, the oral polio vaccine is composed of a live attenuated virus, a non-self antigen, which infects cells and replicates in the vaccinated individual to induce effective immunity against polio virus, a foreign or non-self antigen, without causing clinical disease. Alternatively, the inactivated polio vaccine contains an inactivated or ‘killed’ virus that is incapable of infecting or replicating and is administered subcutaneously to induce protective immunity against polio virus.

DNA therapies have been described for treatment of autoimmune diseases. Such DNA therapies include DNA encoding the antigen-binding regions of the T cell receptor to alter levels of autoreactive T cells driving the autoimmune response (Waisman et al. Nat Med 2:899-905, 1996; U.S. Pat. No. 5,939,400). DNA encoding autoantigens were attached to particles and delivered by gene gun to the skin to prevent MS and collagen induced arthritis. (WO 97/46253; Ramshaw et al. Immunol Cell Biol 75:409-413, 1997). DNA encoding adhesion molecules, cytokines (e.g., TNFα), chemokines (e.g., C—C chemokines), and other immune molecules (e.g., Fas-ligand) have been used for treatment of autoimmune diseases in animal models (Youssef et al. J Clin Invest 106:361-371, 2000; Wildbaum et al. J Clin Invest 106:671-679, 2000; Wildbaum et al. J Immunol 165:5860-5866, 2000).

Methods for treating autoimmune disease by administering a nucleic acid encoding one or more autoantigens are described in WO 00/53019, WO 2003/045316, and WO 2004/047734. While these methods have been successful, further improvements are still needed.

Bacterial enterotoxins are used as immunostimulating adjuvants in vaccines for the prevention of infectious diseases. Cholera toxin (CT) and the closely related E. coli heat-labile toxin (LT) are perhaps the most powerful and best studied mucosal adjuvants in experimental use today (Rappuoli et al. Immunol Today 20:493-500), but when exploited in the clinic their potential toxicity and association with cases of Bell's palsy (paralysis of the facial nerve) have led to their withdrawal from the market (Gluck et al. J Infect Dis 181: 1129-1132, 2000; Gluck et al. Vaccine 20 (Supp1.1): S42-44, 2001; Mutsch et al. N Engl J. Med. 350: 896-903, 2004). The bacterial enterotoxins CT and LT have proven to be effective immunoenhancers in experimental animals as well as in humans. (Freytag et al. Curr Top Microbiol Immunol 236: 215-236, 1999). Structurally these enterotoxins are AB₅ complexes and consist of one ADP-ribosyltransferase active A1 subunit and an A2 subunit that links the A1 to a pentamer of B subunits. The holotoxins bind to most mammalian cells via the B subunit (CTB), which specifically interacts with the GM1-ganglioside receptor in the cell membrane. Whereas the holotoxins have been found to enhance mucosal immune responses, conjugates between CTB and antigen have been used to specifically tolerize the immune system. (Holmgren et al. Am J Trop Med Hyg 50: 42-54, 1994). Studies in mice have shown that CT and LT can accumulate in the olfactory nerve and bulb when given intranasally, a mechanism that is dependent on the ability of the B subunits of CT or LT to bind GM1-ganglioside receptors, present on all nucleated mammalian cells (Fujihashi et al. Vaccine 20: 2431-2438, 2002). Although less toxic mutants of CT and LT have been engineered with substantial adjuvant function, such molecules still carry a significant risk of causing adverse reactions, (Giuliani et al. J Exp Med 187: 1123-1132, 1998; Yamamoto et al. J Exp Med 185: 1203-1210, 1997) especially when considering that the adjuvanticity of CT and LT appears to be a combination of the ADP-ribosyltransferase activity of the A subunit and the ability to bind ganglioside receptors on the target cells (Soriani et al. Microbiology 148: 667-676, 2002). These observations and others preclude the use of CT or LT holotoxins in vaccines for humans. On the other hand, recent observations have demonstrated that it is possible to retain adjuvant functions of these molecules with no toxicity or greatly reduced toxicity by introducing site-directed mutations in the gene coding for the A1 subunit. Examples of mutant molecules that have proven to be effective adjuvants are LTK63 and LTR72, (Giuliani et al. J Exp Med 187: 1123-1132, 1998) the former with no enzymatic activity and the latter with significantly reduced ADP-ribosylating ability. Notwithstanding this, the GM1-ganglioside receptor-dependent binding remains a problem in these mutants and, thus, may still cause nerve cell accumulation and neurotoxicity.

A better solution to this dilemma of efficacy versus toxicity is the CTA1-DD molecule that has proven to be a highly effective mucosal and systemic adjuvant (Agren et al. J Immunol 158: 3936-3946, 1997; U.S. Pat. No. 5,917,026). This unique adjuvant is based on the enzymatically active A1-subunit of CT, combined with a dimer of an immunoglobulin-binding element from Staphylococcus aureus protein A. The molecule thereby avoids binding to all nucleated cells, which could result in unwanted reactions, and exploits fully the CTA1-enzyme in the holotoxin. Accordingly, all studies to date have found that CTA1-DD is nontoxic and has retained excellent immunoenhancing functions. When given systemically, CTA1-DD provides comparable adjuvant effect to that of intact CT, greatly augmenting both cellular and humoral immunity against specific immunogens coadministered with the adjuvant. It also functions as a mucosal adjuvant and should be safe, as it is devoid of the B subunit that is a prerequisite of CT holotoxin toxicity. CTA 1-DD cannot bind to ganglioside receptors; rather, it targets B cells, limiting the CTA1-DD adjuvant to a restricted repertoire of cells that it can interact with. However, the adjuvant effect is not completely dependent on B cells as been shown in strong induction of specific CD4 T cell immunity following intranasal immunizations using the CTA1-DD adjuvant in B-cell deficient mice (Eliasson et al Vaccine 25: 1243-52, 2008, Akhiani et al. Scand J. Immunol. 63: 97-105, 2006).

The adjuvant effect of CTA1-DD was absent in mutants CTA1-E112K-DD and CTA1-R7K-DD, which lack the ADP-ribosylating enzymatic activity (Lycke, Immunol Lett 97: 193-198, 2005).

Wadell and Lycke (FASEB Journal 15(5), A1230, 2001) using an experimental system based on a fusion between CTA1-R7K-DD and a peptide derived from ovalbumin (OVA-p323-339) claimed to have observed a stimulation of tolerance in a splenic CD4 T-cell population following administration of the CTA1-R7K-OVA-DD fusion protein. However, this meeting abstract provides no experimental details and no results, leaving the reader in doubt as to what experiments actually have been performed and which results that were obtained. It is also questionable whether any results obtained in a nonphysiological system using this OVA peptide can be extended to have any relevance to the pathophysiology of an autoimmune or allergic disease, as this OVA peptide is not a peptide associated with an autoimmune or allergic disease.

A conjugate of CTB and a peptide derived from bovine collagen II has been shown to be able to protect mice from developing collagen induced autoimmune ear disease as well as collagen-induced arthritis (Kim et al. Ann Otol Rhinol Laryngol 110: 646-654, 2001; Tarkowski et al. Arthritis Rheum 42: 1628-34, 1999). CTB may, however, not be suited for human use due to its GM1-ganglioside-binding properties and potential neurotoxic effects, as discussed above.

### BRIEF DESCRIPTION OF THE INVENTION

The present invention relates to improved methods and compositions for the prophylaxis, prevention and/or treatment of an autoimmune or allergic disease comprising administration of an immunomodulating complex, the immunomodulating complex being a fusion protein comprising a mutant subunit of bacterial enterotoxin, a peptide capable of binding to a specific cellular receptor, and one or more epitopes associated with the disease. Administration of a therapeutically or prophylactically effective amount of the immunomodulating complex to a subject elicits suppression of an immune response against an antigen associated with the disease, thereby treating or preventing the disease.

The epitope can be an autoimmune epitope when the disease to be treated is an autoimmune disease, the epitope can be an allergy-provoking epitope when the disease to be treated is an allergic disease.

In one embodiment, the invention provides an immunomodulating complex being a fusion protein comprising a mutant subunit of an ADP-ribosylating-subunit of a bacterial enterotoxin. Preferably the ADP-ribosylating-subunit is selected from the A1-subunit of the cholera toxin (CT), the A1-subunit of the E. coli heat labile enterotoxin (LT), the S1 subunit of the Pertussis toxin (PTX), and ADP-ribosylating subunits from Clostridia, Shigella and Pseudomonas toxins. Most preferably the ADP-ribosylating-subunit of a bacterial enterotoxin is selected from the A1-subunit of the cholera toxin (CT), the A1-subunit of the E. coli heat labile enterotoxin (LT), and the S1 subunit of the Pertussis toxin (PTX).

The ADP-ribosylating-subunit of the bacterial enterotoxin is mutated such that the ADP-ribosylating activity of the ADP-ribosylating-subunit is less than 10% of the ADP-ribosylating activity of the corresponding wild-type ADP-ribosylating-subunit, preferably less than 5% of the ADP-ribosylating activity of the corresponding wild-type ADP-ribosylating-subunit, or more preferably less than 1% of the ADP-ribosylating activity of the corresponding wild-type ADP-ribosylating-subunit.

In one preferred embodiment, the fusion protein comprises the CTA1-R7K mutant (SEQ ID NO:1), where amino acid 7, Arginine, in the native CTA1 has been replaced by a Lysine.

In one embodiment, the fusion protein comprises a peptide which specifically binds to a receptor expressed on a cell capable of antigen presentation, especially cells expressing MHC class I or MHC class II antigen. The antigen-presenting cell may be selected from the group consisting of lymphocytes, such as B-lymphocytes, T-cells, monocytes, macrophages, dendritic cells, Langerhans cells, epithelial cells and endothelial cells.

The peptide is a peptide that binds to receptors of the above cells, preferably to an Ig or Fc receptor expressed by said antigen-presenting cell and most preferably to receptors of B-lymphocytes and dendritic cells.

Examples of specific targeting peptides are peptides capable of binding to receptors of:

(i) granulocyte-macrophage colony-stimulating factor (GM-C SF) capable of binding to the GM-CSF receptor α/β heterodimer present on monocytes, neutrophils, eosinophils, fibroblasts and endothelial cells,

(ii) CD4 and CD8 expressed on T cells which together with the T cell receptor (TcR) act as co-receptors for MHC class II and MHC class I molecules, respectively. MHC class I are expressed on most nucleated cells, whereas MHC class II molecules are expressed on dendritic cells, B cells, monocytes, macrophages, myeloid and erythroid precursor cells and some epithelial cells,

(iii) CD 28 and CTLA-4, two homodimeric proteins expressed mainly on T cells which bind to CD80 and CD86B7 expressed on B cells,

(iv) CD40 present mainly on the surface of mature B cells which interact with CD40L (gp39 or CD 154) expressed on T cells,

(v) different isotypes of the Ig heavy chain constant regions which interact with a number of high or low affinity Fc receptors present on mast cells, basophils, eosinophils, platelets, dendritic cells, macrophages, NK cells and B cells,

(vi) complement receptors (CRs), CR1 and CR2, expressed on B-cells have been shown to be important in the generation of normal humoral immune responses, and they likely also participate in the development of autoimmunity,

(vii) C-type lectin receptors (CLRs) like the Dectin-1 expressed on dendritic cells,

(viii) DEC205, an endocytic receptor for antigen uptake and processing expressed at high levels on a subset of dendritic cells,

(ix) CD11c a cell surface receptor for numerous soluble factors and proteins (LPS, fibrinogen, iC3b) found primarily on myeloid cells,

(x) the mannose receptor present on dendritic cells, macrophages an other antigen presenting cells,

(xi) the specific HSP60 receptor present on macrophages.

(xii) CD103 an integrin alpha chain expressed by a subset of dendritic cells.

According to a particularly preferred embodiment of the invention, said peptide is constituted by protein A or a fragment thereof in single or multiple copies, such as one or more D subunits thereof. According to another particularly preferred embodiment of the invention, said peptide is constituted by an antibody fragment, such as a single chain antibody fragment, which specifically binds to a receptor expressed on a cell capable of antigen presentation.

The peptide is preferably such that the resulting fusion protein is in possession of water solubility and capability of targeting the fusion protein to a specific cell receptor different from receptors binding to the native toxin; thereby mediating intracellular uptake of at least said subunit.

The autoantigenic epitopes can be associated with an autoimmune disease, such as insulin-dependent diabetes mellitus (IDDM), multiple sclerosis (MS), systemic lupus erythrematosus (SLE), or rheumatoid arthritis (RA), Sjögrens syndrome (SS).

In some embodiments the autoantigenic epitopes associated with IDDM is an epitope derived from the group consisting of: preproinsulin; proinsulin, insulin, and insulin B chain; glutamic acid decarboxylase (GAD)-65 and -67; tyrosine phosphatase IA-2; islet-specific glucose-6-phosphatase-related protein (IGRP) and islet cell antigen 69 kD.

In some embodiments the autoantigenic epitope associated with MS is an epitope derived from the group consisting of myelin basic protein (MBP), proteolipid protein (PLP), myelin-associated oligodendrocyte basic protein (MOBP), myelin oligodendrocyte glycoprotein (MOG), and myelin-associated glycoprotein (MAG).

In some embodiments the autoantigenic epitope associated with RA is an epitope derived from the group consisting of type I, II, III, IV, V, IX, and XI collagen, GP-39, filaggrin, and fibrin. In one preferred embodiment the epitope is derived from collagen type II, preferably the epitope is the shared immunodominant collagen II peptide comprising amino acids 260-273 (CII260-273).

The allergic epitopes can be associated with allergic asthma, allergic rhinitis, allergic alveolitis, atopic dermatitis, or food hypersensitivity. In some embodiments the allergic epitopes is an epitope derived from a plant pollen, such as Ole e1 allergen from olive pollen, the Cry jI and Cry jII allergen from the Japanese cedar pollen, the timothy grass pollen nPhl p4, or the major birch pollen allergen Bet v1, the mugwort pollen major allergen Art v1, an animal such as the cat allergen Fel d1 or the dog allergen Can f1, the dust mite allergens Der f1, Der p1, Der m1, Blo t4, a fungal antigen such as the Alternaria antigen Alt a1, the Asperigullus antigen Asp f1, the Cladosporium antigens ClA h1 and Cla h2, the Penicillum antigen Pen ch13; or a food allergen such as the chicken egg white allergens Gal d1, Gal d2, and Gal d3, the peanu allergen Ara h2, the soybean allergen Gly m1, Gly m5 and Gly m6, the fish allergen Gad c1, or the shrimp allegen Pen a1.

In one preferred embodiment, the immunomodulating complex is the fusion protein CTA1-R7K-COL-DD (SEQ ID NO:3), where COL is the shared immunodominant collagen II peptide comprising amino acids 260-273 (CII260-273) (SEQ ID NO:4).

The present invention provides methods and compositions for treatment, prophylaxis and/or prevention of an autoimmune disease such as multiple sclerosis, rheumatoid arthritis, insulin-dependent diabetes mellitus, autoimmune uveitis, Behcet's disease, primary biliary cirrhosis, myasthenia gravis, Sjögren's syndrome, pemphigus vulgaris, scleroderma, pernicious anemia, systemic lupus erythematosus (SLE) and Grave's disease comprising administering to a subject an immunomodulating complex according to the invention comprising one or more autoantigenic epitopes associated with the disease.

In certain embodiments the present invention provides improved methods for the treatment, prophylaxis and/or prevention of the autoimmune disease insulin-dependent diabetes mellitus (IDDM) comprising administering to a subject an immunomodulating complex according to the invention comprising one or more autoantigenic epitopes associated with IDDM. In some embodiments the autoantigenic epitopes associated with IDDM is an epitope derived from the group consisting of: preproinsulin; proinsulin, insulin, and insulin B chain; glutamic acid decarboxylase (GAD)-65 and -67; tyrosine phosphatase IA-2; islet-specific glucose-6-phosphatase-related protein (IGRP) and islet cell antigen 69 kD.

In other embodiments of the present invention improved methods are provided for treatment, prohpylaxis and/or prevention of multiple sclerosis (MS) comprising administering to a subject an immunomodulating complex according to the invention comprising one or more autoantigenic epitopes associated with MS. In some embodiments the autoantigenic epitope is an epitope derived from the group consisting of myelin basic protein (MBP), proteolipid protein (PLP), myelin-associated oligodendrocyte basic protein (MOBP), myelin oligodendrocyte glycoprotein (MOG), and myelin-associated glycoprotein (MAG).

In other embodiments improved methods for treatment, prophylaxis and/or prevention of rheumatoid arthritis (RA) are provided comprising administering to a subject an immunomodulating complex according to the invention comprising one or more autoantigenic epitopes associated with RA. In some embodiments the autoantigenic epitope is an epitope derived from the group consisting of type I, II, III, IV, V, IX, and XI collagen, GP-39, filaggrin, and fibrin. In one preferred embodiment the epitope is derived from collagen type II, preferably the epitope is the shared immunodominant collagen II peptide comprising amino acids 260-273 (CII260-273).

According to a particularly preferred embodiment of the invention, said peptide is constituted by protein A or a fragment thereof in single or multiple copies, such as one or more D subunits thereof. According to another particularly preferred embodiment of the invention, said peptide is constituted by an antibody fragment, such as a single chain antibody fragment, which specifically binds to a receptor expressed on a cell capable of antigen presentation.

Multiple immunomodulating complexes comprising different autoantigenic epitopes may be administered as a cocktail, and each individual immunomodulating complex may comprise multiple autoantigenic epitopes. Similarly, multiple immunomodulating complexes comprising different allergic epitopes may be administered as a cocktail, and each individual immunomodulating complex may comprise multiple allergy-provoking epitopes.

In certain variations, the methods and compositions for the treatment, prophylaxis and/or prevention of an autoimmune or allergic disease further comprise the administration of the immunomodulating complex according to the invention in combination with other substances, such as, for example, polynucleotides comprising an immune modulatory sequence, pharmacological agents, adjuvants, cytokines, or vectors encoding cytokines.

Yet another embodiment of the present invention provides a pharmaceutical composition comprising an immunomodulating complex according to the invention. The pharmaceutical composition of the invention can be used for prophylaxis, prevention and/or treatment of an allergic or autoimmune disease. The autoimmune disease can be selected from the group consisting of insulin-dependent diabetes mellitus, multiple sclerosis, rheumatoid arthritis, autoimmune uveitis, primary biliary cirrhosis, myasthenia gravis, Sjögren's syndrome, pemphigus vulgaris, scleroderma, pernicious anemia, systemic lupus erythematosus, and Grave's disease. The allergic disease can be selected from the group consisting of allergic asthma, allergic rhinitis, allergic alveolitis, atopic dermatitis, or food hypersensitivity.

Yet another embodiment of the present invention provides use of an immunomodulating complex according to the invention for the production of a medicinal product for prophylaxis, prevention and/or treatment of an autoimmune or allergic disease. The autoimmune disease can be selected from the group consisting of insulin-dependent diabetes mellitus, multiple sclerosis, rheumatoid arthritis, autoimmune uveitis, primary biliary cirrhosis, myasthenia gravis, Sjogren's syndrome, pemphigus vulgaris, scleroderma, pernicious anemia, systemic lupus erythematosus, and Grave's disease. The allergic disease can be selected from the group consisting of allergic asthma, allergic rhinitis, allergic alveolitis, atopic dermatitis, or food hypersensitivity.

In yet another embodiment the present invention provides isolated nucleic acid sequences encoding an immunomodulating complex according to the invention. Accordingly, the present invention provides isolated nucleic acid sequences encoding an immunomodulating complex being a fusion protein comprising a mutant subunit of bacterial enterotoxin, a peptide capable of binding to a specific cellular receptor, and one or more epitopes associated an autoimmune or allergic disease.

In one embodiment, the nucleic acid according to the invention encodes a fusion protein comprising a mutant subunit of an ADP-ribosylating-subunit of a bacterial enterotoxin. Preferably the v-subunit is selected from the A1-subunit of the cholera toxin (CT), the A1-subunit of the E. coli heat labile enterotoxin (LT), the S1 subunit od the Pertussis toxin (PTX), and ADP-ribosylating subunit of Clostridia, Shigella and Pseudomonas toxins. Most preferably the ADP-ribosylating-subunit of a bacterial enterotoxin is selected from the A1-subunit of the cholera toxin (CT), the A1-subunit of the E. coli heat labile enterotoxin (LT), and the S1 subunit of the Pertussis toxin (PTX). The ADP-ribosylating-subunit of the bacterial enterotoxin is mutated such that the ADP-ribosylating activity of the ADP-ribosylating-subunit is less than 10% of the ADP-ribosylating activity of the corresponding wild-type ADP-ribosylating-subunit, preferably less than 5% of the ADP-ribosylating activity of the corresponding wild-type ADP-ribosylating-subunit, or more preferably less than 1% of the ADP-ribosylating activity of the corresponding wild-type ADP-ribosylating-subunit.

In one embodiment, the nucleic acid according to the invention encodes a fusion protein comprising a peptide which specifically binds to a receptor expressed on a cell capable of antigen presentation, especially cells expressing MHC class I or MHC class II molecules. The antigen-presenting cell may be selected from the group consisting of lymphocytes, such as B-lymphocytes, T-cells, monocytes, macrophages, dendritic cells, Langerhans cells, epithelial cells and endothelial cells.

In one embodiment, the nucleic acid according to the invention encodes a fusion protein comprising an autoantigenic epitope associated with an autoimmune disease, such as insulin-dependent diabetes mellitus (IDDM), multiple sclerosis (MS), systemic lupus erythrematosus (SLE), or rheumatoid arthritis (RA), or Sjögrens syndrome (SS).

In another embodiment, the nucleic acid according to the invention encodes a fusion protein comprising an allergic epitope associated with an allergic disease, such as allergic asthma, allergic rhinitis, allergic alveolitis, atopic dermatitis, or food hypersensitivity.

In some embodiments the autoantigenic epitopes associated with IDDM is an epitope derived from the group consisting of: preproinsulin; proinsulin, insulin, and insulin B chain; glutamic acid decarboxylase (GAD)-65 and -67; tyrosine phosphatase IA-2; islet-specific glucose-6-phosphatase-related protein (IGRP) and islet cell antigen 69 kD. In some embodiments the autoantigenic epitope associated with MS is an epitope derived from the group consisting of myelin basic protein (MBP), proteolipid protein (PLP), myelin-associated oligodendrocyte basic protein (MOBP), myelin oligodendrocyte glycoprotein (MOG), and myelin-associated glycoprotein (MAG). In some embodiments the autoantigenic epitope associated with RA is an epitope derived from the group consisting of type I, II, III, IV, V, IX, and XI collagen, GP-39, filaggrin, and fibrin. In some embodiments the autoantigenic epitope associated with SS is an epitope derived from the group consisting of heat-chock protein HSP60, fodrin, the Ro (or SSA) and the La (or SSB) ribonucleoproteins.

The nucleic acids of the invention can be DNA or RNA.

In another embodiment the invention provides a pharmaceutical composition comprising a nucleic acid according to the invention. The pharmaceutical composition can be used for prophylaxis, prevention and/or treatment of an allergic or autoimmune disease. The invention further provides methods for prophylaxis, prevention and/or treatment of an autoimmune or allergic disease in a subject, the method comprising: administering to the subject an effective amount of a nucleic acid according to the invention.

In yet another embodiment the present invention provides recombinant plasmids, vectors and expression systems comprising a nucleic acid according to the invention. The recombinant expression systems are preferably adapted for bacterial expression. The invention further provides transformed cells containing a plasmid, vector or an expression system according to the invention. The transformed cells are preferably transformed bacterial cells.

### DEFINITIONS

Unless defined otherwise, all technical and scientific terms used herein have the same meaning as commonly understood by those of ordinary skill in the art to which this invention belongs. As used herein, the following terms and phrases have the meanings ascribed to them unless specified otherwise.

The terms “polynucleotide” and “nucleic acid” refer to a polymer composed of a multiplicity of nucleotide units (ribonucleotide or deoxyribomicleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, DNA, synthetic forms, and mixed polymers, both sense and antisense strands, double- or single-stranded, and can also be chemically or biochemically modified or can contain non-natural or derivatized nucleotide bases, as will be readily appreciated by the skilled artisan.

“Antigen,” as used herein, refers to any molecule that can be recognized by the immune system that is by B cells or T cells, or both.

“Autoantigen,” as used herein, refers to an endogenous molecule, typically a polysaccharide or a protein or fragment thereof, that elicits a pathogenic immune response. Autoantigen includes glycosylated proteins and peptides as well as proteins and peptides carrying other forms of post-translational modifications, including citrullinated peptides. When referring to the autoantigen or epitope thereof as “associated with an autoimmune disease,” it is understood to mean that the autoantigen or epitope is involved in the pathophysiology of the disease either by inducing the pathophysiology (i.e., associated with the etiology of the disease), mediating or facilitating a pathophysiologic process; and/or by being the target of a pathophysiologic process. For example, in autoimmune disease, the immune system aberrantly targets autoantigens, causing damage and dysfunction of cells and tissues in which the autoantigen is expressed and/or present. Under normal physiological conditions, autoantigens are ignored by the host immune system through the elimination, inactivation, or lack of activation of immune cells that have the capacity to recognize the autoantigen through a process designated “immune tolerance.”

“Allergen” as used herein, refers to an exogenous molecule, typically a polysaccharide or a protein or fragment thereof, that elicits a pathogenic immune response. Allergen includes glycosylated proteins and peptides as well as proteins and peptides carrying other forms of post-translational modifications. The allergen may be derived from e.g. pollen, fungi, insect venom, dander, mold, foodstuffs. Numerous food allergens are purified and well-characterized, such as peanut Ara h1, Ara h2, Ara h3 and Ara h6; chicken egg white Gal d1, Gal d2, and Gal d3; soybean Gly m1; fish-Gad c1; and shrimp-Pen a1. The major cat (Fel d1) and dog (Can f1) allergens, as well as the dust mite allergens Der f1 and Der p1 are well characterized. The native timothy grass pollen nPhl p4 as well as a number of related recombinant allergens, rPhl 1p, rPhl 2p, rPhl 5p, rPhl 6p, rPhl 7p, rPhl 11p, rPhl 12p, the major birch pollen allergen Bet v1, the major plantain pollen allergen Pla I 1, the major olive pollen allergen Ole e1, the major ragweed pollen allergen Amb a1, the major artemesia pollen allergens Art v1 and Art v3, are well defined.

As used herein the term “epitope” is understood to mean a portion of a polysaccharide or polypeptide having a particular shape or structure that is recognized by either B-cells or T-cells of the animal's immune system. An epitope can include portions of both a polysaccharide and a polypeptide, e.g. a glycosylated peptide.

“Autoantigenic epitope” refers to an epitope of an autoantigen that elicits a pathogenic immune response.

“Allergy-provoking epitope” refers to an epitope of an allergen that elicits a pathogenic immune response

The terms “polypeptide”, “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Self-protein”, “self-polypeptide”, or self-peptide” are used herein interchangeably and refer to any protein, polypeptide, or peptide, or fragment or derivative thereof that: is encoded within the genome of the animal; is produced or generated in the animal; may be modified posttranslationally at some time during the life of the animal; and, is present in the animal non-physiologically. The term “non-physiological” or “non-physiologically” when used to describe the self-protein(s), -polypeptide(s), or -peptide(s) of this invention means a departure or deviation from the normal role or process in the animal for that self-protein, -polypeptide, or -peptide. When referring to the self-protein, -polypeptide or -peptide as “associated with a disease” or “involved in a disease” it is understood to mean that the self-protein, -polypeptide, or -peptide may be modified in form or structure and thus be unable to perform its physiological role or process or may be involved in the pathophysiology of the condition or disease either by inducing the pathophysiology; mediating or facilitating a pathophysiologic process; and/or by being the target of a pathophysiologic process. For example, in autoimmune disease, the immune system aberrantly attacks self-proteins causing damage and dysfunction of cells and tissues in which the self-protein is expressed and/or present. Alternatively, the self-protein, -polypeptide or -peptide can itself be expressed at non-physiological levels and/or function non-physiologically. For example in neurodegenerative diseases self-proteins are aberrantly expressed, and aggregate in lesions in the brain thereby causing neural dysfunction. In other cases, the self-protein aggravates an undesired condition or process. For example in osteoarthritis, self-proteins including collagenases and matrix metalloproteinases aberrantly degrade cartilage covering the articular surface of joints. Examples of posttranslational modifications of self-protein(s), -polypeptide(s) or -peptide(s) are glycosylation, addition of lipid groups, reversible phosphorylation, addition of dimethylarginine residues, citrullination, and proteolysis, and more specifically citrullination of fillagrin and fibrin by peptidyl arginine deiminase (PAD), alpha beta-crystallin phosphorylation, citrullination of MBP, and SLE autoantigen proteolysis by caspases and granzymes. Immunologically, self-protein, -polypeptide or -peptide would all be considered host self-antigens and under normal physiological conditions are ignored by the host immune system through the elimination, inactivation, or lack of activation of immune cells that have the capacity to recognize self-antigens through a process designated “immune tolerance”. A self-protein, -polypeptide, or -peptide does not include immune proteins, polypeptides, or peptides which are molecules expressed physiologically exclusively by cells of the immune system for the purpose of regulating immune function. The immune system is the defence mechanism that provides the means to make rapid, highly specific, and protective responses against the myriad of potentially pathogenic microorganisms inhabiting the animal's world. Examples of immune protein(s), polypeptide(s) or peptide(s) are proteins comprising the T-cell receptor, immunoglobulins, cytokines including the type I interleukins, and the type II cytokines, including the interferons and IL-10, TNF, lymphotoxin, and the chemokines such as macrophage inflammatory protein-1 alpha and beta, monocyte-chemotactic protein and RANTES, and other molecules directly involved in immune function such as Fas-ligand. There are certain immune protein(s), polypeptide(s) or peptide(s) that are included in the self-protein, -polypeptide or -peptide of the invention and they are: class I MHC membrane glycoproteins, class II MHC glycoproteins and osteopontin. Self-protein, -polypeptide or -peptide does not include proteins, polypeptides, and peptides that are absent from the subject, either entirely or substantially, due to a genetic or acquired deficiency causing a metabolic or functional disorder, and are replaced either by administration of said protein, polypeptide, or peptide or by administration of a polynucleotide encoding said protein, polypeptide or peptide (gene therapy). Examples of such disorders include Duchenne' muscular dystrophy, Becker's muscular dystrophy, cystic fibrosis, phenylketonuria, galactosemia, maple syrup urine disease, and homocystinuria.

“Modulation of”, “modulating”, or “altering an immune response” as used herein refers to any alteration of an existing or potential immune responses against an autoimmune or allergy provoking epitope, including, e.g., nucleic acids, lipids, phospholipids, carbohydrates, self-polypeptides, protein complexes, or ribonucleoprotein complexes, that occurs as a result of administration of an immunomodulating complex or polynucleotide encoding an immunomodulating complex. Such modulation includes any alteration in presence, capacity, or function of any immune cell involved in or capable of being involved in an immune response. Immune cells include B cells, T cells, NK cells, NK T cells, professional antigen-presenting cells, non-professional antigen-presenting cells, inflammatory cells, or any other cell capable of being involved in or influencing an immune response. “Modulation” includes any change imparted on an existing immune response, a developing immune response, a potential immune response, or the capacity to induce, regulate, influence, or respond to an immune response. Modulation includes any alteration in the expression and/or function of genes, proteins and/or other molecules in immune cells as part of an immune response.

“Modulation of an immune response” includes, for example, the following: elimination, deletion, or sequestration of immune cells; induction or generation of immune cells that can modulate the functional capacity of other cells such as autoreactive lymphocytes, antigen presenting cells, or inflammatory cells; induction of an unresponsive state in immune cells (i.e., anergy); increasing, decreasing, or changing the activity or function of immune cells or the capacity to do so, including but not limited to altering the pattern of proteins expressed by these cells. Examples include altered production and/or secretion of certain classes of molecules such as cytokines, chemokines, growth factors, transcription factors, kinases, costimulatory molecules, or other cell surface receptors; or any combination of these modulatory events.

For example, administration of an immunomodulating complex or a polynucleotide encoding an immunomodulating complex can modulate an immune response by eliminating, sequestering, or inactivating immune cells mediating or capable of mediating an undesired immune response; inducing, generating, or turning on immune cells that mediate or are capable of mediating a protective immune response; changing the physical or functional properties of immune cells; or a combination of these effects. Examples of measurements of the modulation of an immune response include, but are not limited to, examination of the presence or absence of immune cell populations (using flow cytometry, immunohistochemistry, histology, electron microscopy, polymerase chain reaction (PCR)); measurement of the functional capacity of immune cells including ability or resistance to proliferate or divide in response to a signal (such as using T cell proliferation assays and pepscan analysis based on ³H-thymidine incorporation following stimulation with anti-CD3 antibody, anti-T cell receptor antibody, anti-CD28 antibody, calcium ionophores, PMA, antigen presenting cells loaded with a peptide or protein antigen; B cell proliferation assays); measurement of the ability to kill or lyse other cells (such as cytotoxic T cell assays); measurements of the cytokines, chemokines, cell surface molecules, antibodies and other products of the cells (e.g., by flow cytometry, enzyme-linked immunosorbent assays, Western blot analysis, protein microarray analysis, immunoprecipitation analysis); measurement of biochemical markers of activation of immune cells or signaling pathways within immune cells (e.g., Western blot and immunoprecipitation analysis of tyrosine, serine or threonine phosphorylation, polypeptide cleavage, and formation or dissociation of protein complexes; protein array analysis; DNA transcriptional, profiling using DNA arrays or subtractive hybridization); measurements of cell death by apoptosis, necrosis, or other mechanisms (e.g., annexin V staining, TUNEL assays, gel electrophoresis to measure DNA laddering, histology; fluorogenic caspase assays, Western blot analysis of caspase substrates); measurement of the genes, proteins, and other molecules produced by immune cells (e.g., Northern blot analysis, polymerase chain reaction, DNA microarrays, protein microarrays, 2-dimensional gel electrophoresis, Western blot analysis, enzyme linked immunosorbent assays, flow cytometry); and measurement of clinical symptoms or outcomes such as improvement of autoimmune, neurodegenerative, and other diseases involving self proteins or self polypeptides (clinical scores, requirements for use of additional therapies, functional status, imaging studies) for example, by measuring relapse rate or disease severity (using clinical scores known to the ordinarily skilled artisan) in the case of multiple sclerosis, measuring blood glucose in the case of type I diabetes, or joint inflammation in the case of rheumatoid arthritis.

“Subjects” shall mean any animal, such as, for example, a human, non-human primate, horse, cow, dog, cat, mouse, rat, guinea pig or rabbit.

“Treating”, “treatment”, or “therapy” of a disease or disorder shall mean slowing, stopping or reversing the disease's progression, as evidenced by decreasing, cessation or elimination of either clinical or diagnostic symptoms, by administration of an immunomodulating complex or a polynucleotide encoding an immunomodulating complex, either alone or in combination with another compound as described herein. “Treating”, “treatment”, or “therapy” also means a decrease in the severity of symptoms in an acute or chronic disease or disorder or a decrease in the relapse rate as for example in the case of a relapsing or remitting autoimmune disease course or a decrease in inflammation in the case of an inflammatory aspect of an autoimmune disease. In the preferred embodiment, treating a disease means reversing or stopping or mitigating the disease's progression, ideally to the point of eliminating the disease itself. As used herein, ameliorating a disease and treating a disease are equivalent.

“Preventing”, “prophylaxis”, or “prevention” of a disease or disorder as used in the context of this invention refers to the administration of an immunomodulating complex or a polynucleotide encoding an immunomodulating complex, either alone or in combination with another compound as described herein, to prevent the occurrence or onset of a disease or disorder or some or all of the symptoms of a disease or disorder or to lessen the likelihood of the onset of a disease or disorder.

A “therapeutically or prophylactically effective amount” of an immunomodulating complex refers to an amount of the immunomodulating complex that is sufficient to treat or prevent the disease as, for example, by ameliorating or eliminating symptoms and/or the cause of the disease. For example, therapeutically effective amounts fall within broad range(s) and are determined through clinical trials and for a particular patient is determined based upon factors known to the skilled clinician, including, e.g., the severity of the disease, weight of the patient, age, and other factors.

### DESCRIPTION OF THE DRAWINGS

FIG. 1. DNA Construct Encoding the Immunomodulating Complex CTA1-R7K-COL-DD

The pCTA1-DD plasmid contains the cholera toxin A1 gene (aa 1-194) cloned at HindIII-BamHI and two D fragments from the staphylococcal protein A gene under the control of the trp promoter. Collagen peptide was inserted between the CTA1 and the DD fragment to give pCTA1-COL-DD. The R7K mutation was constructed by in vitro mutagenesis giving pCTA1-R7K-COL-DD. Ptr=trp promoter. COL=collagen peptide, D=Ig-binding element from S. aureus protein A.

FIG. 2. The ADP Ribosyltransferase Activity

The ADP ribosyltransferase activity of the CT, CTA1-DD and CTA1-R7K-COL-DD were assayed for ADP-ribosylagmatine formation though incorporation of [U-14C]adenine. The values represent mean cpm.

FIG. 3. IgG Binding

The ability of CTA1-R7K-COL-DD to bind to human IgG1 on solid phase was determined by ELISA. Briefly, 96-well plates were coated over night with 10 μg/ml in PBS at room temperature and thereafter washed and blocked with 5% BSA/PBS. Serial dilutions of CTA1-R7K-COL-DD were incubated in corresponding subwells. After 2 h wells were washed extensively and phosphatase-labeled rabbit anti-mouse IgG at 1/100 dilution was added to each well. Substrate was added and the binding of CTA1-R7K-COL-DD to the human IgG1 was detected by enzymatic reaction and assessed as OD at 450 nm using a spectrophotometer.

FIG. 4. Intranasal Administration with Inactive or Active CTA1-COL-DD Adjuvant.

DBA/1 mice received 5 μg of CTA1-COL-DD or CTA1-R7K-COL-DD intranasally. Control mice received PBS. One week later all mice were given a challenge ip. with the collagen protein in Ribi-adjuvant. Mice were sacrificed 16 days after intranasal administration to assess the level of collagen-specific T cell responses to recall antigen in vitro. Proliferation was assessed after 72 h. of culturing and determined as the level of incorporated [3H] TcR uptake per well. The data is presented as mean c.p.m±SD. Representative results from two experiments with 5 mice per group.

FIG. 5. Intranasal Administration with Inactive and Active CTA1-COL-DD Adjuvant.

DBA/1 mice were administered with 5 μg of CTA1-COL-DD or CTA1-R7K-COL-DD intra nasally. Control mice received PBS. One week later all mice were given a challenge ip. with collagen in Ribi-adjuvant and following another 8 days the level of collagen-specific T cell responses to recall antigen were assessed in vitro. Cytokine (IFN-γ) production were measured in culture supernatants from cells stimulated for 96 h and expressed as mean cytokine concentrations in ng/ml±SD above background levels from cultures with cells from untreated mice. Representative results from three experiments with 5 mice per group.

FIG. 6. Induction of Local Tolerance in Draining Lymph Nodes

DBA/1 recipients were given PBS or 5 μg of CTA1-COL-DD or CTA1-R7K-COL-DD. One week later all mice were immunized i.p with collagen in Ribi-adjuvant. Mice were sacrificed 16 days after the intranasal treatment and T cell proliferation was determined in cervical lymph nodes (CLN). Proliferative response were recorded at 72 h. of culturing, and assessed by [3H] TcR uptake and given as mean c.p.m±SD. One representative experiment out of three with five mice per group.

FIG. 7. Inhibition of Anti-Collagen Type II Antibody Production.

DBA/1 recipients were given PBS or 5 μg of CTA1-COL-DD or CTA1-R7K-COL-DD. One week later all mice received an i.p challenge immunization with collagen plus Ribi-adjuvant Collagen specific total IgG and IgA titers were measured by ELISA. A) IgA titer. B) IgG titer. Results are representative of three experiments with five animals per group and values are given as mean log10 titers±s.e.m.

FIG. 8. Mucosal Treatment with CTA1-R7K-COL-DD

For induction of collagen induced arthritis (CIA), rat collagen type II (CII) emulsified with Freund's complete adjuvant was injected into the tail of the mice. At 21 days later, CII emulsified with Freunds incomplete adjuvant was injected into the tail as a booster to elicit disease in the joints. Mice were treated i.n prophylactically as well as therapeutically and the degree of joint tissue affection and destruction was monitored and assessed at 42 days following elicitation of disease. Animals were treated i.n with PBS, CTA1-R7K-DD or CTA1-R7K-COL-DD on three consecutive days before or after the collagen immunizations. A) Frequency of arthritis over time. B) Frequency of arthritis at day 45. C) Arthritis score at day 45.

FIG. 9. The Effects of CTA1-R7K-COL-DD on Joint at the Histological Level.

The joints of CIA control (A) (PBS) and CTA1-R7K-COL-DD (B) treated mice were removed and fixed in formalin and stained with hematoxylin and eosin. One low and one high power image of CIA-joints is shown and cell infiltration and cartilage/bone destructions are clearly visible.

FIG. 10. Histological Changes After Mucosal CTA1-R7K-COL-DD Treatment of DBA/1 Mice

The joints of CIA control (PBS) and CTA1R7K-COL-DD treated mice. Joints were removed and fixed in formalin and stained with hematoxylin and eosin. The histological micrographs were scored in blind by two independent investigators and the mean results of the grades are given.

FIG. 11. Greatly Augmented IL-10 and Reduced IL-6 Production in CTA1R7K-COL-DD Treated CIA Mice

Serum was collected at sacrifice from untreated (PBS) (□) CIA mice or from mice treated with 5 μg of CTA1R7K-DD ( [symbol] ) or CTA1R7K-COL-DD (▪) and analyzed for the concentration of IL-10 (A), IL-6 (B). The cytokine levels are given as mean pg/ml±SD of 10-12 mice per group. This is one representative experiment of two giving similar results. P-values indicate significance compared to the results in untreated control CIA-mice.

FIG. 12. Skewing of CII-Specific CD4 T Cell Responses Towards Regulatory T Cells and IL-10

Spleen lymphocytes were isolated from untreated (PBS) (□) CIA mice or from mice treated with 5 μg of CTA1R7K-DD ( [symbol] ) or CTA1R7K-COL-DD (▪) and stimulated in vitro in the presence or absence of recall COL-peptide. Supernatants were harvested after 96 h and analyzed for the contents of IL-10 (A) and IL-6 (B). Values are given as mean pg/ml±SD for groups of 10-12 mice in each experiment. Results are the mean of 2 independent experiments giving similar results

### DETAILED DESCRIPTION OF THE INVENTION

The present invention relates to methods and compositions for the prophylaxis, prevention and/or treatment of a disease in a subject associated with one or more self-protein(s), -polypeptide(s), or -peptide(s) present in the subject and involved in a non-physiological state. The invention is more particularly related to methods and compositions for the prophylaxis, prevention and/or treatment of autoimmune diseases associated with one or more self-polypeptide(s) present in a subject in a non-physiological state such as in multiple sclerosis, rheumatoid arthritis, insulin dependent diabetes mellitus, autoimmune uveitis, primary biliary cirrhosis, myasthenia gravis, Sjögren's syndrome, pemphigus vulgaris, scleroderma, pernicious anemia, systemic lupus erythematosus and Grave's disease. The present invention provides improved methods for the prophylaxis, prevention and/or treatment of an autoimmune disease comprising administering to a subject an immunomodulating complex comprising one or more autoantigenic epitopes associated with the disease. Administration of a therapeutically or prophylactically effective amount of the immunomodulating complex comprising one or more autoantigenic epitopes to a subject elicits suppression of an immune response against an autoantigen associated with the autoimmune disease, thereby treating or preventing the disease.

### Autoimmune Diseases

Several examples of autoimmune diseases associated autoantigens are set forth in Table 1, and particular examples are described in further detail herein below.

TABLE 1
Exemplary Autoimmune Diseases and Associated Autoantigens

| | | Autoantigen(s) Associated with the |
|---|---|---|
| Autoimmune Disease | Tissue Targeted | Autoimmune Disease |
| Rheumatoid Arthritis | synovial joints | Immunoglobulin, fibrin, filaggrin, type I, II, III, |
| | | IV, V, IX, and XI collagens, GP-39, hnRNPs |
| Multiple sclerosis | central nervous system | myelin basic protein, proteolipid protein, myelin |
| | | associated glycoprotein, cyclic nucleotide |
| | | phosphodiesterase, myelin-associated |
| | | glycoprotein, myelin-associated |
| | | oligodendrocytic basic protein, myelin |
| | | oligodendrocyte glycoprotein, alpha-B-crystalin |
| Insulin Dependent | Dependent β cells in | tyrosine phosphatase IA2, IA-2β; glutamic acid |
| Diabetes Mellitus | islets of pancreas | decarboxylase (65 and 67 kDa forms), |
| | | carboxypeptidase H, insulin, proinsulin, pre- |
| | | proinsulin, heat shock proteins, glima 38, islet |
| | | cell antigen 69 KDa, p52, islet cell glucose |
| | | transporter GLUT-2 |
| Sjögrens Syndrome | Exocrine glands | heat chock protein HSP60, fodrin, |
| | | ribonuceloproteins Ro60 (SSA), Ro52 (SSA), |
| | | and La (SSB), poly(ADP-ribose) polymerase, |
| | | lipocalin, alpha amylase |
| Guillian Barre | peripheral nervous | peripheral myelin protein I and others |
| Syndrome | system | |
| Autoimmune Uveitis | eye, uvea | S-antigen, interphotoreceptor retinoid binding |
| | | protein (IRBP), rhodopsin, recoverin |
| Primary Biliary | biliary tree of liver | pyruvate dehydrogenase complexes (2-oxoacid |
| Cirrhosis | | dehydrogenase) |
| Autoimmune Hepatitis | Liver | Hepatocyte antigens, cytochrome P450 |
| Pemphigus vulgaris | Skin | Desmoglein-1, -3, and others |
| Myasthenia Gravis | nerve-muscle junctions | acetylcholine receptor |
| Autoimmune gastritis | stomach/parietal cell | H⁺/K⁺ ATPase, intrinsic factor |
| Pernicious Anemia | Stomach | intrinsic factor |
| Polymyositis | Muscle | histidyl tRNA synthetase, other synthetases, |
| | | other nuclear antigens |
| Autoimmune | Thyroid | Thyroglobulin, thyroid peroxidase |
| Thyroiditis | | |
| Graves's Disease | Thyroid | Thyroid-stimulating hormone receptor |
| Psoriasis | Skin | Unknown |
| Vitiligo | Skin | Tyrosinase, tyrosinase-related protein-2 |
| Systemic Lupus Eryth | Systemic | nuclear antigens: DNA, histones,. |
| | | ribonucleoproteins |
| Celiac Disease | Small bowel | Transglutaminase |

Rheumatoid Arthritis. Rheumatoid arthritis (RA) is a chronic autoimmune inflammatory synovitis affecting 0.8% of the world population. It is characterized by chronic inflammatory synovitis that causes erosive joint destruction. RA is mediated by T cells, B cells and macrophages.

Evidence that T cells play a critical role in RA includes the (1) predominance of CD4⁺ T cells infiltrating the synovium, (2) clinical improvement associated with suppression of T cell function with drugs such as cyclosporine, and (3) the association of RA with certain HLA-DR alleles. The HLA-DR alleles associated with RA contain a similar sequence of amino acids at positions 67-74 in the third hypervariable region of the β chain that are involved in peptide binding and presentation to T cells. RA is mediated by autoreactive T cells that recognize a self-protein, or modified self-protein, present in synovial joints. Autoantigens that are targeted in RA comprise, e.g., epitopes from type II collagen; hnRNP; A2/RA33; Sa; filaggrin; keratin; citrulline; cartilage proteins including gp39; collagens type IS III, IV, V, IX, XI; HSP-65/60; IgM (rheumatoid factor); RNA polymerase; hnRNP-B1; hnRNP-D; cardiolipin; aldolase A; citrulline-modified filaggrin and fibrin. Autoantibodies that recognize filaggrin peptides containing a modified arginine residue (de-iminated to form citrulline) have been identified in the serum of a high proportion of RA patients. Autoreactive T and B cell responses are both directed against the same immunodominant type II collagen (CII) peptide 257-270 in some patients.

Multiple Sclerosis. Multiple sclerosis (MS) is the most common demyelinating disorder of the CNS and affects 350,000 Americans and one million people worldwide. Onset of symptoms typically occurs between 20 and 40 years of age and manifests as an acute or sub-acute attack of unilateral visual impairment, muscle weakness, paresthesias, ataxia, vertigo, urinary incontinence, dysarthria, or mental disturbance (in order of decreasing frequency). Such symptoms result from focal lesions of demyelination which cause both negative conduction abnormalities due to slowed axonal conduction, and positive conduction abnormalities due to ectopic impulse generation (e.g., Lhermitte's symptom). Diagnosis of MS is based upon a history including at least two distinct attacks of neurologic dysfunction that are separated in time, produce objective clinical evidence of neurologic dysfunction, and involve separate areas of the CNS white matter. Laboratory studies providing additional objective evidence supporting the diagnosis of MS include magnetic resonance imaging (MRI) of CNS white matter lesions, cerebral spinal fluid (CSF) oligoclonal banding of IgG, and abnormal evoked responses. Although most patients experience a gradually progressive relapsing remitting disease course, the clinical course of MS varies greatly between individuals and can range from being limited to several mild attacks over a lifetime to fulminant chronic progressive disease. A quantitative increase in myelin-autoreactive T cells with the capacity to secrete IFN-gamma is associated with the pathogenesis of MS and EAE.

The autoantigen targets of the autoimmune response in autoimmune demyelinating diseases, such as multiple sclerosis and experimental autoimmune encephalomyelitis (EAE), may comprise epitopes from proteolipid protein (PLP); myelin basic protein (MBP); myelin oligodendrocyte glycoprotein (MOG); cyclic nucleotide phosphodiesterase (CNPase); myelin-associated glycoprotein (MAG)5 and myelin-associated oligodendrocytic basic protein (MBOP); alpha-B-crystallin (a heat shock protein); viral and bacterial mimicry peptides, e.g., influenza, herpes viruses, hepatitis B virus, etc.; OSP (oligodendrocyte specific-protein); citrulline-modified MBP (the C8 isoform of MBP in which 6 arginines have been de-imminated to citrulline), etc. The integral membrane protein PLP is a dominant autoantigen of myelin. Determinants of PLP antigenicity have been identified in several mouse strains, and include residues 139451, 103-116, 215-232, 43-64 and 178-191. At least 26 MBP epitopes have been reported (Meinl et al, J Clin Invest 92, 2633-43, 1993). Notable are residues 1-11, 59-76 and 87-99. Immunodominant MOG epitopes that have been identified in several mouse strains include residues 1-22, 35-55, 64-96.

In human MS patients the following myelin proteins and epitopes were identified as targets of the autoimmune T and B cell response. Antibody eluted from MS brain plaques recognized myelin basic protein (MBP) peptide 83-97 (Wucherpfennig et al. J Clin Invest 100:1114-1122, 1997). Another study found approximately 50% of MS patients having peripheral blood lymphocyte (PBL) T cell reactivity against myelin oligodendrocyte glycoprotein (MOG) (6-10% control), 20% reactive against MBP (8-12% control), 8% reactive against PLP (0% control), 0% reactive MAG (0% control). In this study 7 of 10 MOG reactive patients had T cell proliferative responses focused on one of 3 peptide epitopes, including MOG 1-22, MOG 34-56, MOG 64-96 (Kerlero de Rosbo et al. Eur J Immunol 27: 3059-69, 1997). T and B cell (brain lesion-eluted Ab) response focused on MBP 87-99 (Oksenberg et al. Nature 362: 68-70, 1993). In MBP 87-99, the amino acid motif HFFK is a dominant target of both the T and B cell response (Wucherpfennig et al. J Clin Invest 100: 1114-22, 1997). Another study observed lymphocyte reactivity against myelin-associated oligodendrocytic basic protein (MOBP), including residues MOBP 21-39 and MOBP 37-60 (HoIz et al. J Immunol 164: 1103-9, 2000). Using immunogold conjugates of MOG and MBP peptides to stain MS and control brains both MBP and MOG peptides were recognized by MS plaque-bound Abs (Genain and Hauser, Methods 10: 420-34, 1996).

Insulin Dependent Diabetes Mellitus. Human type I or insulin-dependent diabetes mellitus (IDDM) is characterized by autoimmune destruction of the β cells in the pancreatic islets of Langerhans. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2).

Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration.

The Non-Obese Diabetic (NOD) mouse is an animal model with many clinical, immunological, and histopathological features in common with human IDDM. NOD mice spontaneously develop inflammation of the islets and destruction of the beta cells, which leads to hyperglycemia and overt diabetes. Both CD4⁺ and CD8⁺ T cells are required for diabetes to develop, although the roles of each remain unclear. It has been shown that administration of insulin or GADS as proteins, under tolerizing conditions to NOD mice prevents disease and down-regulates responses to the other autoantigens.

The presence of combinations of autoantibodies with various specificities in serum are highly sensitive and specific for human type I diabetes mellitus. For example, the presence of autoantibodies against GAD and/or IA-2 is approximately 98% sensitive and 99% specific for identifying type I diabetes mellitus from control serum. In non-diabetic first degree relatives of type I diabetes patients, the presence of autoantibodies specific for two of the three autoantigens including GAD, insulin and IA-2 conveys a positive predictive value of >90% for development of type IDM within 5 years.

Autoantigens targeted in human insulin dependent diabetes mellitus may include, for example, tyrosine phosphatase IA-2; IA-2[beta]; glutamic acid decarboxylase (GAD) both the 65 kDa and 67 kDa forms; carboxypeptidase H; insulin; proinsulin; heat shock proteins (HSP); glima 38; islet cell antigen 69 KDa (ICA69); p52; two ganglioside antigens (GT3 and GM2-1); islet-specific glucose-6-phosphatase-related protein (IGRP); and an islet cell glucose transporter (GLUT 2).

Human IDDM is currently treated by monitoring blood glucose levels to guide injection, or pump-based delivery, of recombinant insulin. Diet and exercise regimens contribute to achieving adequate blood glucose control.

Autoimmune Uveitis. Autoimmune uveitis is an autoimmune disease of the eye that is estimated to affect 400,000 people, with an incidence of 43,000 new cases per year in the U.S. Autoimmune uveitis is currently treated with steroids, immunosuppressive agents such as methotrexate and cyclosporine, intravenous immunoglobulin, and TNFα-antagonists.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Autoantigens targeted by the autoimmune response in human autoimmune uveitis may include S-antigen, interphotoreceptor retinoid binding protein (IRBP), rhodopsin, and recoverin.

Primary Billiary Cirrhosis. Primary Biliary Cirrhosis (PBC) is an organ-specific autoimmune disease that predominantly affects women between 40-60 years of age. The prevalence reported among this group approaches 1 per 1,000. PBC is characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjögren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al. Immunol Rev 174:210-225, 2000; Mackay et al. Immunol Rev 174:226-237, 2000). AMA soon became a cornerstone for laboratory diagnosis of PBC, present in serum of 90-95% patients long before clinical symptoms appear. Autoantigenic reactivities in the mitochondria were designated as M1 and M2. M2 reactivity is directed against a family of components of 48-74 kDa. M2 represents multiple autoantigenic subunits of enzymes of the 2-oxoacid dehydrogenase complex (2-OADC) and is another example of the self-protein, -polypeptide, or -peptide of the instant invention. Studies identifying the role of pyruvate dehydrogenase complex (PDC) antigens in the etiopathogenesis of PBC support the concept that PDC plays a central role in the induction of the disease (Gershwin et al. Immunol Rev 174:210-225, 2000; Mackay et al. Immunol Rev 174:226-237, 2000). The most frequent reactivity in 95% of cases of PBC is the E2 74 kDa subunit, belonging to the PDC-E2. There exist related but distinct complexes including: 2-oxoglutarate dehydrogenase complex (OGDC) and branched-chain (BC) 2-OADC. Three constituent enzymes (E1,2,3) contribute to the catalytic function which is to transform the 2-oxoacid substrate to acyl co-enzyme A (CoA), with reduction of NAD to NADH. Mammalian PDC contains—an additional component, termed protein X or E-3 Binding protein: (E3BP). In PBC patients, the, major antigenic response is directed against PDC-E2 and E3BP. The E2 polypeptide contains two tandemly repeated lipoyl domains, while E3BP has a single lipoyl domain. The lipoyl domain is found in a number of autoantigen targets of PBC and is referred to herein as the “PBC lipoyl domain.” PBC is treated with glucocorticoids and immunosuppressive agents including methotrexate and cyclosporin A.

Sjögren's Syndrome. Sjögren's syndrome (SS) is a chronic autoimmune disease, which affects primarily salivary and lacrimal glands leading to dry eyes (keratoconjunctivitis sicca) and dry mouth (xerostomia). Other organs, which may be involved, include the bronchial tree, kidneys, liver, blood vessels, peripheral nerves and the pancreas. Of particular interest is the dual presentation of SS: either alone as primary disorder in women of the fourth and fifth decades (primary SS) or in the context of other autoimmune diseases (secondary SS); glandular (sicca symptoms) and systemic (extraglandular) clinical manifestations may be present. Characteristics of SS is the presence of rheumatoid factors, antinuclear and precipitating autoantibodies. The cytoplasmic/nuclear ribonucleoprotein particles (Ro/SSA and La/SSB) have a prominent role in the autoimmune response of SS. Other antigens involved in the positive nuclear pattern by immunofluorescence include the following: Ku, NOR-90 (nucleolar organizing region), p-80 coilin, HMG-17 (high-mobility group), Ki/SL. Furthermore, organ-specific autoantibodies are also recognized including antithyroglobulin, antierythrocyte and antisalivary gland epithelium antibodies. (Reviewed in Clio et al. Int Arch Allergy Immunol 123:46-57, 200). A 120-kD organ-specific autoantigen has been identified as the cytoskeletal protein α-fodrin (Haneji et al. Science 276:604-607, 1997). HSP60 is another autoantigen suggested to be involved in SS Immunization with HSP60 or a HSP60-derived peptide (amino acid residues 437-460) have been shown to reduce SS-related histopathologic features in an animal model of SS (Dalaleu et al. Arthritis Rheum 58:2318-2328, 2008). The major target antigens Ro/SSA, La/SSB and their cognate antibodies have been extensively defined at the molecular level. Ro/SSA is a ribonucleoprotein containing small, cytoplasmic RNAs. The protein component of the Ro/SSA antigen, a 60-kD protein (60-kD Ro/SSA, Ro60) is bound to one of several small cytoplasmic RNA molecules. A 52-kD peptide is another component of Ro/SSA antigen (52-kD Ro/SSA; Ro52). La/SSB antigen is composed of a polypeptide consisting of 408 amino acids. Both 60-kD Ro/SSA and La/SSB proteins are members of a family of RNA-binding proteins that contain a sequence of 80 amino acids known as the RNA recognition motif (RNP). B cell epitope mapping of 60-kD Ro/SSA, 52-kD Ro/SSA and La/SSB molecules using several strategies have revealed specific epitopes in several studies. B cell epitopes of 60-kD Ro/SSA autoantigen appear to be located in the central region and the carboxy-terminal part of the molecule. Two disease-specific epitopes: the TKYKQRNGWSHKDLLRSHLKP (169-190) and the ELYKEKALSVETEKLLKYLEAV (211-232) region have been identified (Routsias et al. Eur J Clin Invest 26:514-521, 1996). The antigenic determinants of 52-kD Ro/SSA protein are mainly linear and are found in the central part of the molecule. Four peptides (amino acids 2-11, 107-126, 277-292 and 365-382) have been reported to be recognized by anti-Ro/SSA sera (Ricchiuti et al. Clin Exp Immunol 95:397-407, 1994). Four highly reactive peptides with purified IgG, spanning the regions 145-164, 289-308, 301-320 and 349-368 of the La/SSB protein, have been reported (Tzioufas et al. Clin Exp Immunol 108:191-198, 1997).

Other Autoimmune Diseases And Associated Autoantigens. Autoantigens for myasthenia gravis may include epitopes within the acetylcholine receptor. Autoantigens targeted in pemphigus vulgaris may include desmoglein-3. The dominant autoantigen for pemphigus vulgaris may include desmoglein-3. Panels for myositis may include tRNA synthetases (e.g., threonyl, histidyl, alanyl, isoleucyl, and grycyl); Ku; ScI; SSA; Ul Sn ribonuclear protein; Mi-I; Mi-I; Jo-I; Ku; and SRP. Panels for scleroderma may include Scl-70; centromere; Ul ribonuclear proteins; and fibrillarin. Panels for pernicious anemia may include intrinsic factor; and glycoprotein beta subunit of gastric H/K ATPase. Epitope Antigens for systemic lupus erythematosus (SLE) may include DNA; phospholipids; nuclear antigens; Ro; La; Ul ribonucleoprotein; Ro60 (SS-A); Ro52 (SS-A); La (SS-B); calreticulin; Grp78; Scl-70; histone; Sm protein; and chromatin, etc. For Grave's disease epitopes may include the Na⁺/1-symporter; thyrotropin receptor; Tg; and TPO.

Graft Versus Host Disease. One of the greatest limitations of tissue and organ transplantation in humans is rejection of the tissue transplant by the recipient's immune system. It is well established that the greater the matching of the MHC class I and II (HLA-A, HLA-B, and HLA-DR) alleles between donor and recipient the better the graft survival. Graft versus host disease (GVHD) causes significant morbidity and mortality in patients receiving transplants containing allogeneic hematopoietic cells. Hematopoietic cells are present in bone-marrow transplants, stem cell transplants, and other transplants. Approximately 50% of patients receiving a transplant from a HLA-matched sibling will develop moderate to severe GVHD, and the incidence is much higher in non-HLA-matched grafts. One-third of patients that develop moderate to severe GVHD will die as a result. T lymphocytes and other immune cell in the donor graft attack the recipients' cells that express polypeptides variations in their amino acid sequences, particularly variations in proteins encoded in the major histocompatibility complex (MHC) gene complex on chromosome 6 in humans. The most influential proteins for GVHD in transplants involving allogeneic hematopoietic cells are the highly polymorphic (extensive amino acid variation between people) class I proteins (HLA-A, -B, and -C) and the class II proteins (DRB1, DQB1, and DPB1) (Appelbaum, Nature 411, 385-389, 2001). Even when the MHC class I alleles are serologically ‘matched’ between donor and recipient, DNA sequencing reveals there are allele-level mismatches in 30% of cases providing a basis for class I-directed GVHD even in matched donor-recipient pairs (Appelbaum, Nature 411, 385-389, 2001). The minor histocompatibility self-antigens GVHD frequently causes damage to the skin, intestine, liver, lung, and pancreas. GVHD is treated with glucocorticoids, cyclosporine, methotrexate, fludarabine, and OKT3.

Tissue Transplant Rejection Immune rejection of tissue transplants, including lung, heart, liver, kidney, pancreas, and other organs and tissues, is mediated by immune responses in the transplant recipient directed against the transplanted organ. Allogeneic transplanted organs contain proteins with variations in their amino acid sequences when compared to the amino acid sequences of the transplant recipient. Because the amino acid sequences of the transplanted organ differ from those of the transplant recipient they frequently elicit an immune response in the recipient against the transplanted organ. Rejection of transplanted organs is a major complication and limitation of tissue transplant, and can cause failure of the transplanted organ in the recipient. The chronic inflammation that results from rejection frequently leads to dysfunction in the transplanted organ. Transplant recipients are currently treated with a variety of immunosuppressive agents to prevent and suppress rejection. These agents include glucocorticoids, cyclosporin A, Cellcept, FK-506, and OKT3.

### Compositions and Methods for Treatment

The present invention provides improved methods and compositions for the treatment, prophylaxis and/or prevention of an autoimmune or allergic disease comprising an immunomodulating complex comprising one or more epitopes associated with the disease. The immunomodulating complex according to the present invention comprises one or more epitopes associated with the autoimmune or allergic disease. The improved method of the present invention includes the administration of an immunomodulating complex comprising one or more epitopes associated with the disease.

In certain embodiments the present invention provides improved methods for the treatment, prophylaxis and/or prevention of the autoimmune disease insulin-dependent diabetes mellitus (IDDM) comprising administering to a subject an immunomodulating complex comprising one or more autoantigenic epitopes associated with IDDM.

The immunomodulating complex administered to treat or prevent IDDM may include autoimmune epitopes derived from one or more of self-proteins, for example preproinsulin, proinsulin, glutamic acid decarboxylase (GAD)-65 and -67; tyrosine phosphatase IA-2; islet-specific glucose-6-phosphatase-related protein (IGRP); and/or islet cell antigen 69 kD. Alternatively the immunomodulating complex administered to treat or prevent IDDM may include multiple autoimmune epitopes derived form the same or different self-protein(s), -polypeptide(s), or -peptide(s). In preferred embodiments the immunomodulating complex administered to treat or prevent IDDM may include autoimmune epitopes derived the self-polypeptide preproinsulin or proinsulin.

In other embodiments of the present invention improved methods are provided for the treatment, prophylaxis and/or prevention of multiple sclerosis (MS) comprising administering to a subject an immunomodulating complex comprising one or more autoantigenic epitopes associated with MS. The immunomodulating complex administered to treat MS may include an autoantigen epitope derived from one or more self-polypeptides including but not limited to: myelin basic protein (MBP), myelin oligodendrocyte glycoprotein (MOG), proteolipid protein (PLP)5 myelin-associated oligodendrocytic basic protein (MOBP), myelin oligodendrocyte glycoprotein (MOG), and/or myelin-associated glycoprotein (MAG). Alternatively, an immunomodulating complex comprising multiple autoantigenic epitopes derived form the same or different self-protein(s), -polypeptide(s), or -peptide(s) associated with the disease.

In other embodiments of the present invention improved methods for treatment, prophylaxis and/or prevention of rheumatoid arthritis (RA) are provided comprising administering to a subject an immunomodulating complex according to the invention comprising one or more autoantigenic epitopes associated with RA. In some embodiments the autoantigenic epitope is an epitope derived from the group consisting of type I, II, III, IV, V, IX, and XI collagen, GP-39, filaggrin, and fibrin. In one preferred embodiment the epitope is derived from collagen type II, preferably the epitope is the shared immunodominant collagen II peptide comprising amino acids 260-273 (CII260-273).

Alternatively multiple immunomodulating complexes comprising autoantigenic epitopes derived from different self-polypeptides may be administered.

In yet another embodiment the present invention provides nucleic acid sequences, including DNA and RNA sequences, encoding the immunomodulating complexes according the invention as well as plasmid, vectors and expression systems comprising such nucleic acid sequences.

The immunomodulating complexes according to the invention can be produced by recombinant DNA technology.

Techniques for construction of plasmids, vectors and expression systems and transfection of cells are well-known in the art, and the skilled artisan will be familiar with the standard resource materials that describe specific conditions and procedures.

Construction of the plasmids, vectors and expression system of the invention employs standard ligation and restriction techniques that are well-known in the art (see generally, e.g., Ausubel, et al, Current Protocols in Molecular Biology, Wiley Interscience, 1989; Sambrook and Russell, Molecular Cloning, A Laboratory Manual 3rd ed. 2001). Isolated plasmids, DNA sequences, or synthesized oligonucleotides are cleaved, tailored, and relegated in the form desired. Sequences of DNA constructs can be confirmed using, e.g., standard methods for DNA sequence analysis (see, e.g., Sanger et al. (1977) Proc. Natl. Acad. Sci., 74, 5463-5467).

Yet another convenient method for isolating specific nucleic acid molecules is by the polymerase chain reaction (PCR) (Mullis et al. Methods Enzymol 155:335-350, 1987) or reverse transcription PCR (RT-PCR). Specific nucleic acid sequences can be isolated from RNA by RT-PCR. RNA is isolated from, for example, cells, tissues, or whole organisms by techniques known to one skilled in the art. Complementary DNA (cDNA) is then generated using poly-dT or random hexamer primers, deoxynucleotides, and a suitable reverse transcriptase enzyme. The desired polynucleotide can then be amplified from the generated cDNA by PCR. Alternatively, the polynucleotide of interest can be directly amplified from an appropriate cDNA library. Primers that hybridize with both the 5′ and 3′ ends of the polynucleotide sequence of interest are synthesized and used for the PCR. The primers may also contain specific restriction enzyme sites at the 5′ end for easy digestion and ligation of amplified sequence into a similarly restriction digested plasmid vector.

### Delivery of Immunomodulating Complexes

Therapeutically and prophylactically effective amounts of an immunomodulating complex are in the range of about 1 μg to about 10 mg. A preferred therapeutic or prophylactically effective amount of an immunomodulating complex is in the range of about 5 μg to about 1 mg. A most preferred therapeutic amount of immunomodulating complex is in the range of about 10 μg to 100 μg. In certain embodiments, the immunomodulating complex is administered monthly for 6-12 months, and then every 3-12 months as a maintenance dose. Alternative treatment regimens may be developed and may range from daily, to weekly, to every other month, to yearly, to a one-time administration depending upon the severity of the disease, the age of the patient, the immunomodulating complex being administered, and such other factors as would be considered by the ordinary treating physician.

In one embodiment, the immunomodulating complex is delivered intranasally. In other variations, the immunomodulating complex is delivered orally, sublingually, subcutaneously, transcutaneous, intradermally, intravenously, mucosally or intramuscularly.

### Formulation

The immunomodulating complex can be administered in combination with other substances, such as, for example, pharmacological agents, adjuvants, cytokines, or immune stimulating complexes (ISCOMS).

### EXAMPLES

The following examples are specific embodiments for carrying out the present invention. The examples are offered for illustrative purposes only, and are not intended to limit the scope of the present invention in any way.

### Example 1

### Immunomodulating Complex CTA1-R7K-COL-DD

Construction of CTA1-DD mutants, expression and purification of fusion proteins were performed essentially as described by Agren (J Immunol 1999, 162: 2432-2440).

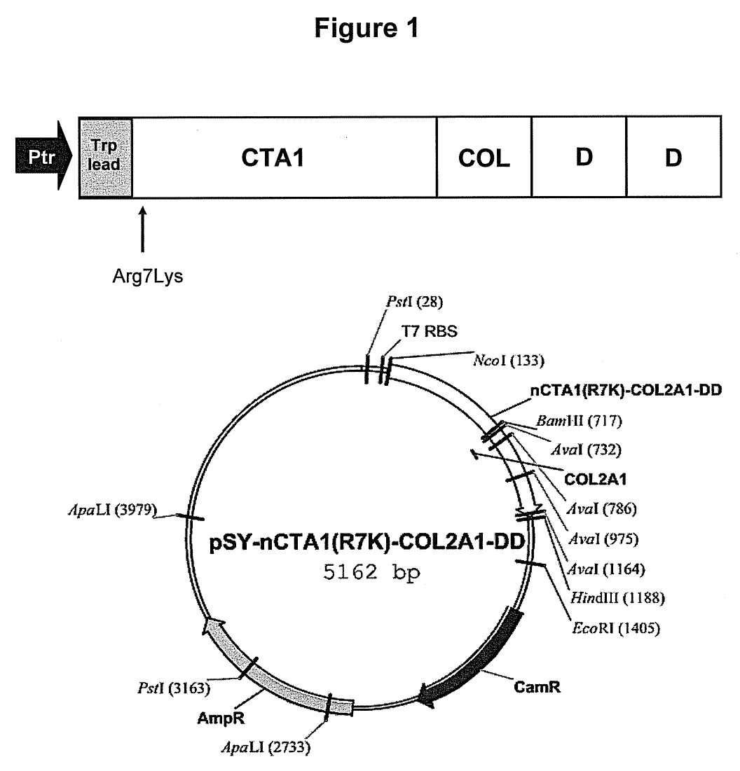

The pCTA1-DD plasmid contains the cholera toxin A1 gene (aa 1-194) cloned at HindIII-BamHI and DNA coding two D fragments from the staphylococcal protein A gene under the control of the trp promoter. DNA encoding a collagen peptide, the shared immunodominant collagen II peptide (CII260-273), was inserted between DNA encoding the CTA1 and the DD moieties giving the pCTA1-R7K-COL-DD plasmid. (FIG. 1).

### Example 2

### ADP Ribosylating Activity

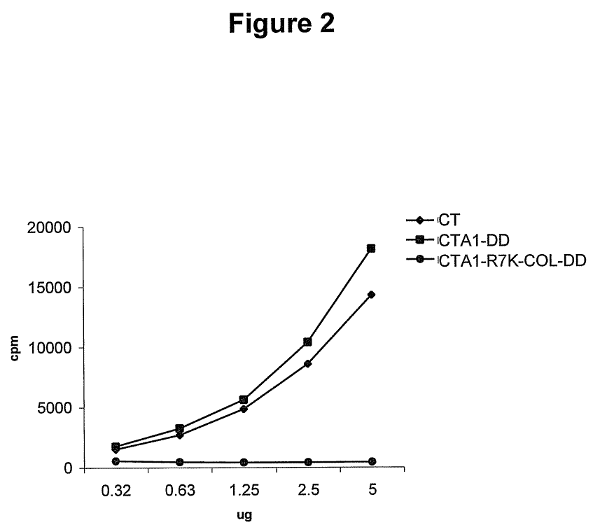

It was investigated whether the changes in molecular design had functional consequences for the enzymatic activity of CTA1. The ADP ribosyltransferase activity was analyzed using the cell-free NAD:agmatine-assay. A linear dose response activity of CTA1-COL-DD was found. By contrast, no ADP-riboylating activity was found with CTA1-R7K-COL-DD (FIG. 2). These results clearly demonstrated that CTA1-R7K-DD had lost its enzymatic activity.

### Example 3

### Binding to IgG

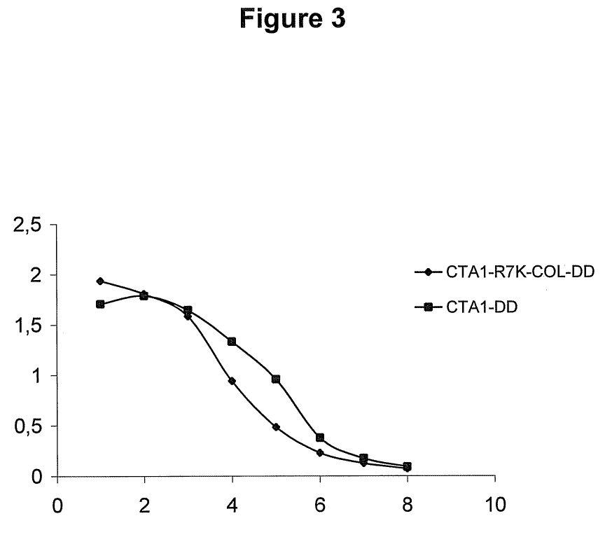

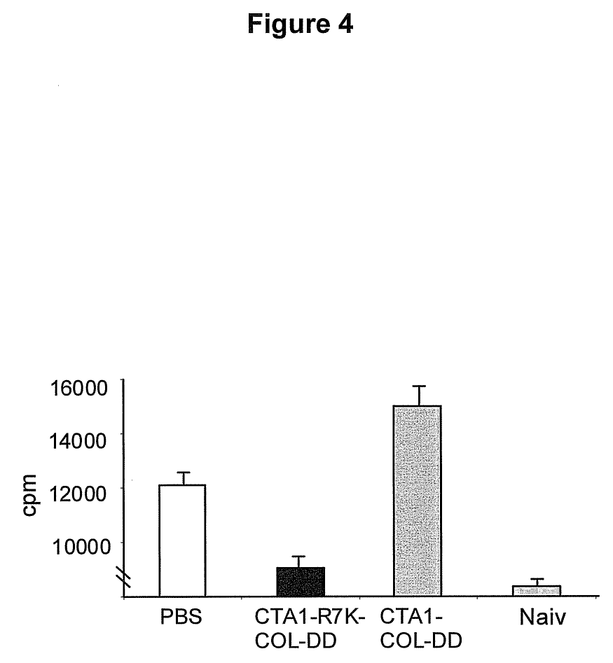

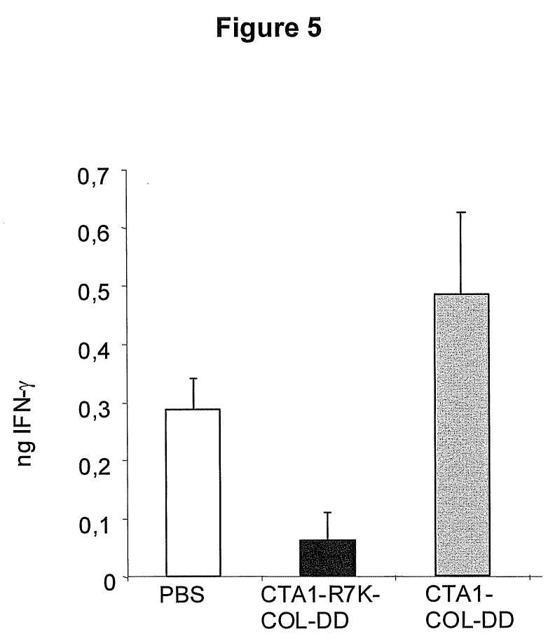

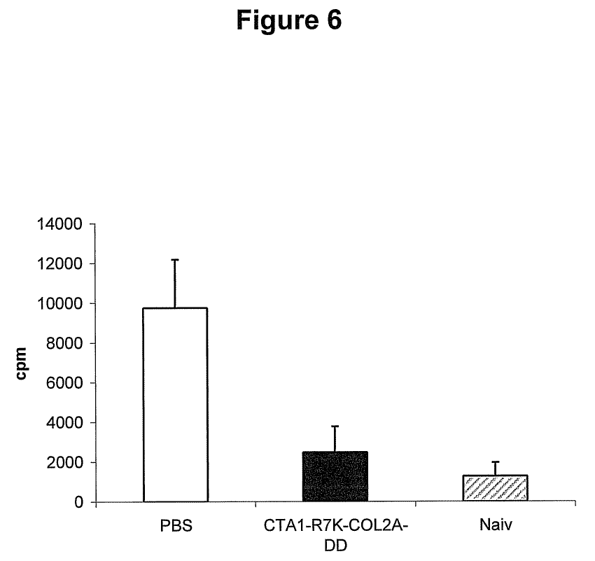

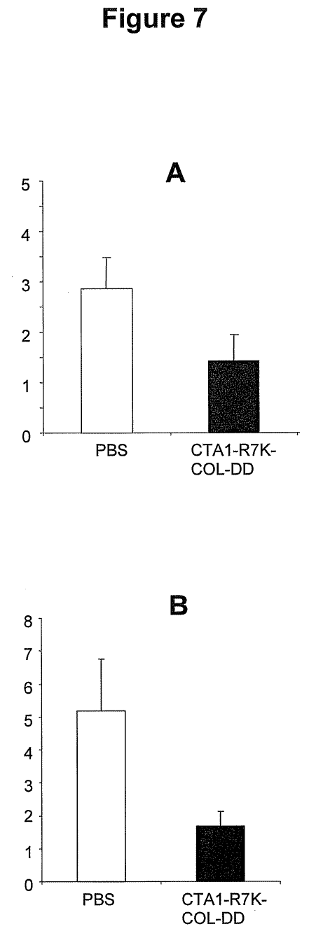

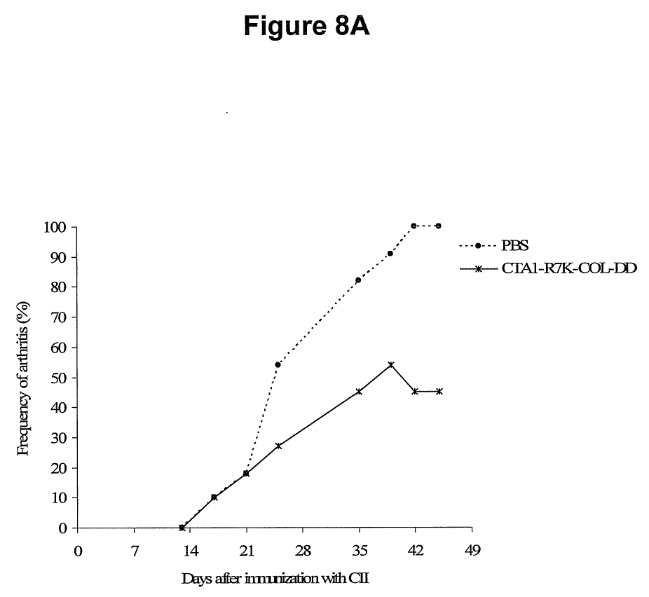

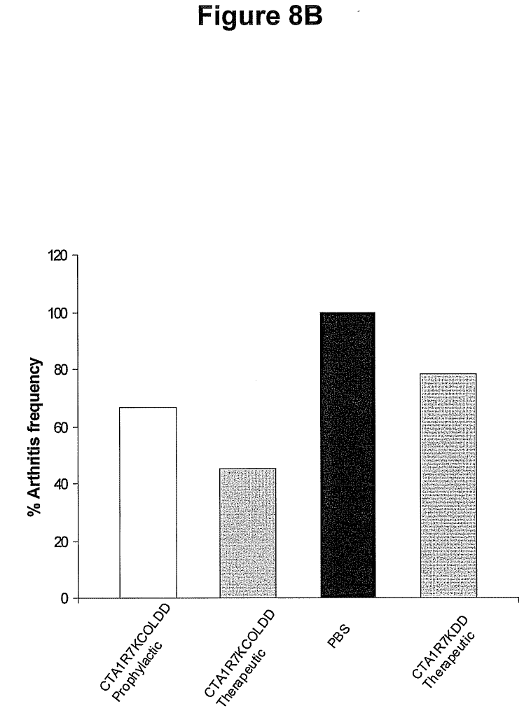

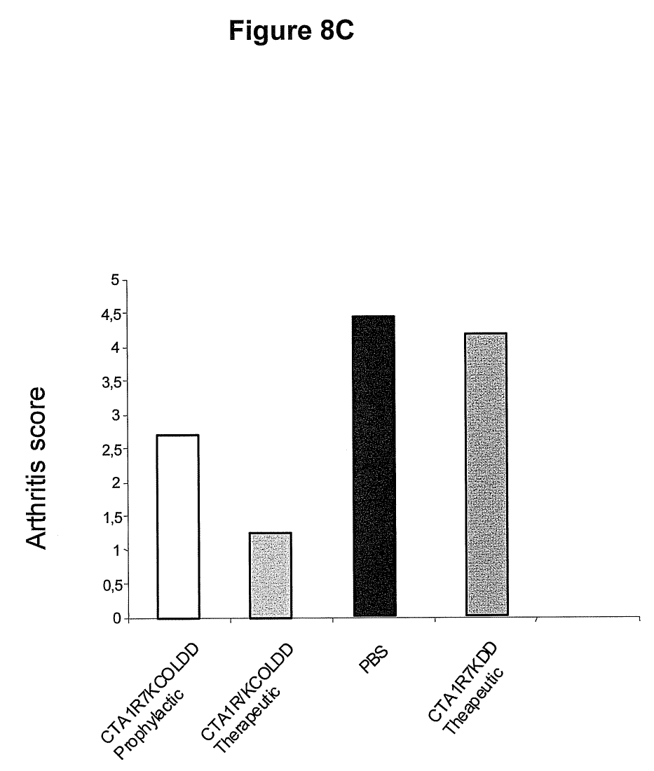

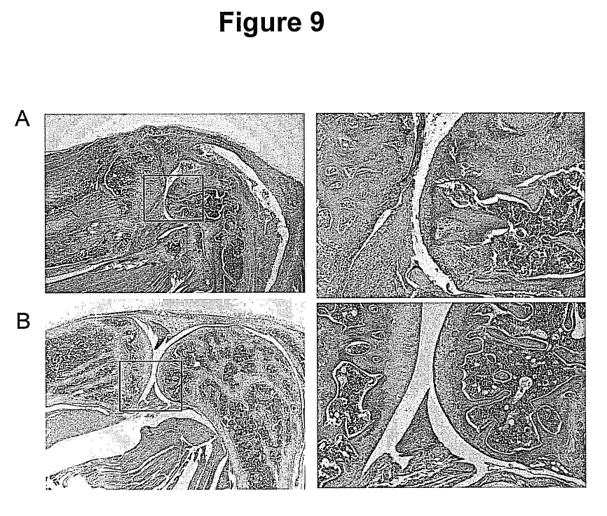

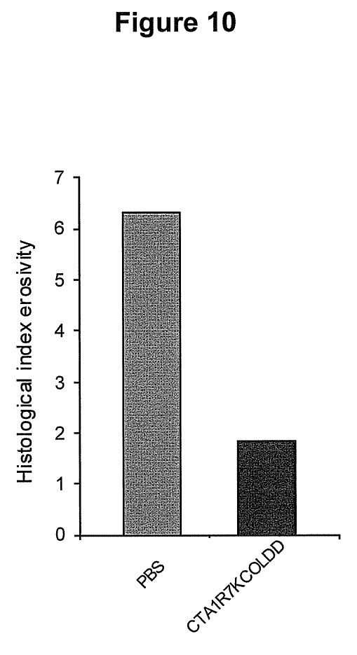

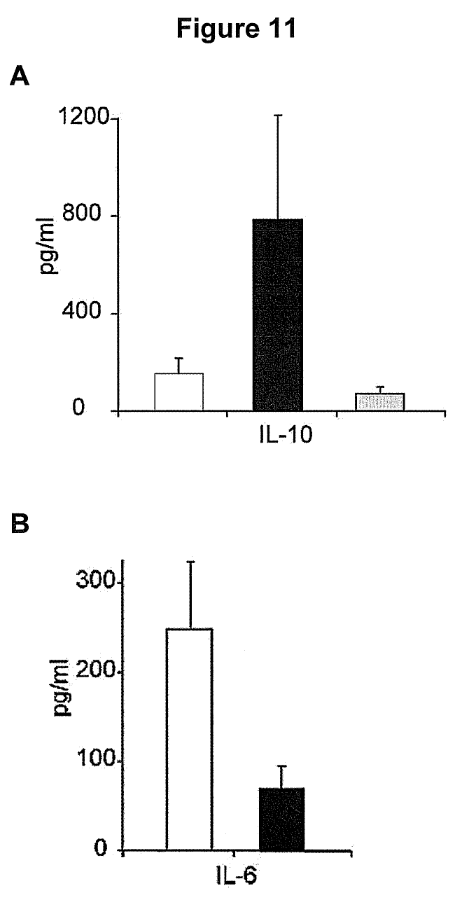

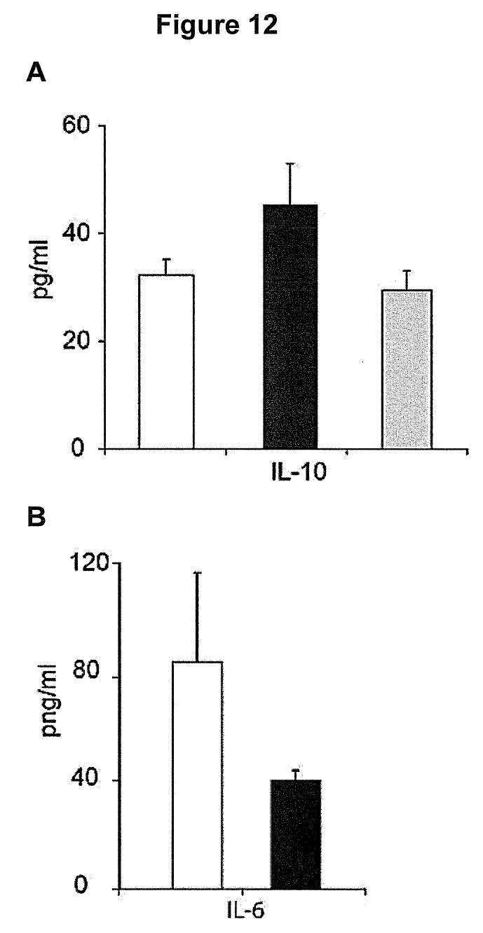

IgG binding was measured by ELISA. The CTA1-R7K-COL-DD mutant has retained its ability to bind to human IgG in solid phase, indicating that the DD-element was unaffected by the mutation in CTA1 (FIG. 3).

### Example 4

### Intranasal Administration with Inactive or Active CTA1-Col-DD Adjuvant

The CTA1R7K-COL-DD mutant was used to stimulate T cell tolerance in vivo. DBA/1 mice received 5 μg of CTA1-COL-DD or CTA1-R7K-COL-DD intranasally. Control mice received PBS. One week later all mice were given a challenge ip. with the collagen protein in Ribi-adjuvant. Mice were sacrificed 16 days after intranasal administration to assess the level of collagen-specific T cell responses to recall antigen in vitro. CD4⁺ T cell recall responses to peptide in vitro were investigated. Cells were isolated from spleen and subjected to re-stimulation with COL or whole collagen protein. It was found that the T-cell proliferative response to collagenII (CII) was significantly lower in cells isolated from spleens of CTA1-R7K-COL-DD treated mice than in cells from untreated (PBS) control mice (FIG. 4). By contrast, the enzymatically active CTA1-COL-DD fusion protein induced strong enhancement of T cell priming as evident from the greatly augmented proliferative responses to recall antigen exposure in vitro (FIG. 4). The enzymatically inactive construct demonstrated consistently impaired T cell responses in vivo following intranasal administration. No adverse reactions to administration of the CTA1-R7K-COL-DD was recorded; no effect on the mean body weight, nor did it affect behaviour or local inflammatory reactions at the site of application. Thus, CTA1-R7K-COL-DD appears to be a safe and non-toxic means of promoting specific T cell tolerance.

### Example 5

### Reduced IFN-γ Production in Tolerized T Cells After Nasal Administration of CTA1-R7K-COL-DD

To examine the effect of intranasal CTA1-R7K-COL-DD administration on the cytokine activity of immune T cells in response to recall antigen exposure production of IFN-γ in supernatants were measured. Reduced levels of IFN-γ T cells from mice treated with the CTA1R7K-COL-DD tolerogen were observed. By contrast, mice given the active CTA1-COL-DD were much stronger producers of IFN-γ than untreated control mice (FIG. 5). Therefore, the decreased production of IFN-γ to recall antigen after intranasal treatment confirmed that mice were effectively tolerized by CTA1-R7K-COL-DD.

### Example 6

### Tolerance Following Intranasal Treatment with CTA1-R7K-COL-DD

To determine whether the systemic tolerance detected in the splenic T cells also was induced in T cells in draining lymph nodes mice were treated with CTA1-R7K-COL-DD intranasally and one weak later mice were sacrificed and lymphocytes from the cervical lymph nodes were prepared. It was found T cell responses to recall Ag were strongly suppressed (FIG. 6).

### Example 7

### Inhibition of Anti-Collagen Type II Antibody Production

To get a better understanding of the extent of tolerance induced by CTA1-R7K-COL-DD treatment serum responses to the challenge immunization following intranasal treatments were analyzed. DBA/1 recipients were given PBS or 5 μg of CTA1-COL-DD or CTA1-R7K-COL-DD. One week later all mice received an i.p challenge immunization with collagen plus Ribi-adjuvant. Collagen specific total IgG and IgA titers were measured by ELISA. Anti-collagen responses were strongly reduced and both anti-collagen IgG and IgA were reduced several-fold (FIG. 7).

### Example 8

### Treatment of CIA in Mice

The mouse CIA model of RA was used to determine the clinical value of intranasal treatments with the CTA1-R7K-COL-DD tolerogen. The CIA model shares a number of clinical, histologic, and immunologic features with RA, and it is therefore the most used model to test potential therapeutic agents against RA. DBA1 mice were treated intranasally with PBS, CTA1-R7K-DD or CTA1—R7K-COL-DD before or after a challenge immunization with collagen in Freund's complete adjuvant (FCA) followed by a booster with Freund's incomplete adjuvant (IFA) on day 21. Mice were then sacrificed to determine the incidence of arthritis and arthritis articular index of CIA.

The therapeutic effect of CTA1-R7K-COL-DD-treatment of mice resulted in decrease in the incidence (FIGS. 8A, 8B) and severity (FIG. 8C) of CIA as compared with control group (PBS), as assessed by the paw swelling and clinical score. After treatment with CTA1-R7K-COL-DD on day 26, 27 and 28 much less swelling was noted.

The arthritis index in the control PBS group increased dramatically three weeks after the collagen-immunizations and reached a peak at 6 weeks. By contrast, in the CTA1-R7K-COL-DD group significantly lower arthritis index were scored and many animals had no symptoms at all. At completion of the experiment only 40% of the mice had developed arthritis in the group treated with CTA1-R7K-COL-DD, whereas 100% of the control mice were affected. Moreover, the arthritis index revealed that of the mice scored positive for arthritis in the treated group a majority of the mice were less afflicted with disease (FIG. 8C). The control mice treated with CTA1-R7K-DD, without peptide, were as affected as the PBS control mice, indicating that it was the COL-specific effect that prevented disease and not the CTA1-R7K-DD carrier (FIG. 8B). Interestingly, both therapeutic and prophylactic treatments with CTA1-R7K-COL-DD had significant protective effects.

### Example 9

### CTA1-R7K-COL-DD Prevents Histological Changes in the CIA Mouse Model

The arthritis scoring data were further confirmed by histological analysis of specimens taken after CTA1-R7K-COL-DD treatments. Mice were sacrificed and joints were fixed in formalin and stained with hematoxylin and eosin. Cartilage erosion and synovial cell infiltration and destruction of cartilage and bone were more severe in untreated mice (FIG. 9A). By contrast, CTA1-R7K-COL-DD treated mice demonstrated significantly reduced or no signs of disease (FIG. 9B). Histological sections from mice confirmed that CTA1-R7K-COL-DD treatment completely prevented or significantly reduced disease compared to untreated control mice that showed 100% afflicted joints with severe tissue destruction. Importantly, destruction of bone and cartilage was significantly lower in the CTA1-R7K-COL-DD treated compared to the control mice (FIG. 10). Noteworthy, no significant differences in weight were observed during the course of these experiments (data not shown). These histopathology results clearly demonstrated that mucosal treatment with CTA1-R7K-COL-DD can effectively suppress the immune pathologic process in the mouse CIA model of RA.

### Example 10

### Greatly Augmented IL-10 and Reduced IL-6 Production in CTA1R7K-COL-DD Treated CIA Mice

Serum was collected at sacrifice from untreated (PBS) CIA mice or from mice treated with 5 μg of CTA1R7K-DD or CTA1R7K-COL-DD and analyzed for the concentration of IL-10 (FIG. 11A) and IL-6 (FIG. 11B). Strikingly, it was found that serum IL-10 levels in treated mice were substantially elevated above those detected in untreated or CTA1R7K-DD-treated mice (FIG. 11A). On the other hand, serum contained significantly lower levels of IL-6 in therapeutically treated mice as compared to untreated CIA-diseased mice (FIG. 11B). In the CTA1R7K-DD group similar levels of IL-6 were observed as in untreated mice (not shown).

Furthermore, the CTA1R7K-COL-DD-treatment resulted in significantly reduced anti-CII specific IgG1, IgG2a, IgG2b and IgG3 serum titers as opposed to the levels detected in untreated CIA-diseased mice. Thus, high IL-10 and low IL-6 serum concentrations in individual mice correlated well with the CTA1R7K-COL-DD-induced protection against CIA-disease, as assessed by a low arthritic index.

### Example 11

### Skewing of CH-Specific CD4 T Cell Responses Towards Regulatory T Cells and IL-10

Spleen lymphocytes were isolated from untreated (PBS) CIA mice or from mice treated with 5 μg of CTA1R7K-DD or CTA1R7K-COL-DD and stimulated in vitro in the presence or absence of recall COL-peptide. Supernatants were harvested after 96 h and analyzed for the contents of IL-10 (FIG. 12A) and IL-6 (FIG. 12B).

Dramatically increased serum IL-10 and increased IL-10 production by splenic T cells to a MHC class II-restricted recall peptide-challenge (COL) have observed in CTA1R7K-COL-DD treated mice. Indeed, this suggested a T cell origin of the cytokine and, thus, the induction of regulatory T cells. Several previous studies in the CIA-model have demonstrated that oral tolerance can induce IL-10 producing CD4⁺ regulatory T cells that were FoxP3⁺ CD25⁺ (16, 46). In yet other studies of mucosal tolerance the regulatory T cells have been found to be CD4⁺ CD25⁻ FoxP3⁻ cells producing IL-10. Thus, both natural CD25⁺ and inducible CD25⁻ regulatory T cells may be involved in curbing CIA and perhaps RA. Preliminary studies (not shown) further suggest that i.n treatment with CTA1R7K-OVA-DD promotes such CD4⁺ CD25⁻ FoxP3⁻ Tr1-type of cells. It is therefore concluded that therapeutic i.n CTA1R7K-COL-DD treatment stimulates Treg development, which controls CD4⁺ effector T cell functions, including Th1 and Th17 cells, and, thereby, also suppresses leukocyte infiltration into the synovium, effectively preventing CIA.

1-28. (canceled)

29. An immunomodulating complex being a fusion protein comprising:
(a) a mutant subunit of a bacterial enterotoxin;
(b) a peptide capable of binding to a specific cellular receptor; and
(c) one or more epitopes associated with an autoimmune or allergic disease.

30. The immunomodulating complex according to claim 29, wherein the one or more epitopes are autoimmune epitopes associated with an autoimmune disease.

31. The immunomodulating complex according to claim 30, wherein the autoimmune disease is selected from insulin-dependent diabetes mellitus, multiple sclerosis, rheumatoid arthritis, autoimmune uveitis, primary biliary cirrhosis, myasthenia gravis, Sjögren's syndrome, pemphigus vulgaris, scleroderma, pernicious anemia, systemic lupus erythematosus, and Grave's disease.

32. The immunomodulating complex according to claim 29, wherein the one or more epitopes are allergy-provoking epitopes associated with an allergy-provoking disease.

33. The immunomodulating complex according to claim 32, wherein the allergic disease is selected from allergic asthma, allergic rhinitis, atopic dermatitis and food hypersensitivity.

34. The immunomodulating complex according to claim 29, wherein the fusion protein comprises a mutant subunit of an ADP-ribosylating-subunit of a bacterial enterotoxin.

35. The immunomodulating complex according to claim 34, wherein the ADP-ribosylating-subunit is selected from the A1-subunit of the cholera toxin (CT), the A1-subunit of the E. coli heat labile enterotoxin (LT), the S1 subunit of the Pertussis toxin (PTX), and ADP-ribosylating subunits of Clostridia, Shigella and Pseudomonas toxins.

36. The immunomodulating complex according to claim 35, wherein the ADP-ribosylating-subunit is selected from the A1-subunit of the cholera toxin (CT), the A1-subunit of the E. coli heat labile enterotoxin (LT), and the S1 subunit of the Pertussis toxin (PTX).

37. The immunomodulating complex according to claim 36, wherein the mutant subunit of a bacterial enterotoxin is CTA1-R7K SEQ ID NO:1.

38. The immunomodulating complex according to claim 34, wherein the ADP-ribosylating-subunit of the bacterial enterotoxin is mutated such that the ADP-ribosylating activity of the ADP-ribosylating-subunit is less than 10% of the ADP-ribosylating activity of the corresponding wild-type ADP-ribosylating-subunit, preferably less than 5% of the ADP-ribosylating activity of the corresponding wild-type ADP-ribosylating-subunit, or more preferably less than 1% of the ADP-ribosylating activity of the corresponding wild-type ADP-ribosylating-subunit.

39. The immunomodulating complex according to claim 29, wherein the fusion protein comprises a peptide which specifically binds to a receptor expressed on a cell capable of antigen presentation.

40. The immunomodulating complex according to claim 39, wherein the fusion protein comprises a peptide which specifically binds to a receptor expressed on a cell expressing MHC class I or MHC class II molecules.

41. The immunomodulating complex according to claim 40, wherein the fusion protein comprises a peptide which specifically binds to a receptor expressed on a cell selected from the group consisting of lymphocytes, such as B-lymphocytes, T-cells, monocytes, macrophages, dendritic cells, Langerhans cells, epithelial cells and endothelial cells.

42. The immunomodulating complex according to claim 41, wherein, said peptide is constituted by protein A or a fragment thereof in single or multiple copies, such as one or more D subunits thereof.

43. The immunomodulating complex CTA1-R7K-COL-DD SEQ ID NO:3.

44. A method for prophylaxis, prevention and/or treatment of an autoimmune or allergic disease in a subject, the method comprising: administering to the subject an effective amount of an immunomodulating complex according to claim 29.

45. The method according to claim 44, wherein the autoimmune disease is selected from insulin-dependent diabetes mellitus, multiple sclerosis, rheumatoid arthritis, autoimmune uveitis, primary biliary cirrhosis, myasthenia gravis, Sjögren's syndrome, pemphigus vulgaris, scleroderma, pernicious anemia, systemic lupus erythematosus, and Grave's disease.

46. The method according to claim 44, wherein the allergic disease is selected from allergic asthma, allergic rhinitis, atopic dermatitis and food hypersensitivity.


SEQUENCE LISTING (4 sequences)

SEQ ID NO: 1 - protein, 194 aa, Vibrio cholerae
  VARIANT (7)..(7): R7K MUTATION
NDDKLYKADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSS

SEQ ID NO: 2 - DNA, 1050 bp, Artificial
  SYNTHETHIC FUSION PROTEIN
atggatgataagttatataaggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgggatccgggaagacacccgagggtattgctggcttcaaaggtgaacaaggccccaagggagaacctggccccgaggctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaacgttttaggtgaagctaaaaaattaaacgaatctcaagcacccaaacccgaggctgatgcgcaacaaaataacttcaacaaagatcaacaaagcgccttctatgaaatcttgaacatgcctaacttaaacgaagcgcaacgtaacggcttcattcaaagtcttaaagacgacccaagccaaagcactaacgttttaggtgaagctaaaaaattaaacgaatctcaagcacccaaacccgaggtagcaggtcagaat

SEQ ID NO: 3 - protein, 350 aa, Artificial
  Synthetic Construct
MDDKLYKADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSGSGKTPEGIAGFKGEQGPKGEPGPEADAQQNNFNKDQQSAFYEILNMPNLNEAQRNGFIQSLKDDPSQSTNVLGEAKKLNESQAPKPEADAQQNNFNKDQQSAFYEILNMPNLNEAQRNGFIQSLKDDPSQSTNVLGEAKKLNESQAPKPEVAGQN

SEQ ID NO: 4 - protein, 14 aa, Artificial
  SYNTHETIC PEPTIDE
IAGFKGEQGPKGEP